SEQUENCE LISTING (52 sequences)

SEQ ID NO: 1 - protein, 6 aa, Homo sapiens
  SITE (1)..(6): CDR1 of Light Chain(1656)
QGISNY

SEQ ID NO: 2 - protein, 3 aa, Homo sapiens
  SITE (1)..(3): CDR2 of Light Chain(1656)
SAS

SEQ ID NO: 3 - protein, 9 aa, Homo sapiens
  SITE (1)..(9): CDR3 of Light Chain(1656)
QHLKTSLVT

SEQ ID NO: 4 - protein, 13 aa, Homo sapiens
  SITE (1)..(13): CDR1 of Heavy Chain(1656)
GFTDSRFTFRNYW

SEQ ID NO: 5 - protein, 8 aa, Homo sapiens
  SITE (1)..(8): CDR2 of Heavy Chain(1656)
INGDGGGT

SEQ ID NO: 6 - protein, 19 aa, Homo sapiens
  SITE (1)..(19): CDR3 of Heavy Chain(1656)
VREFGLFDSGTWYSGGFDY

SEQ ID NO: 7 - protein, 106 aa, Homo sapiens
  SITE (1)..(106): Variable Region of Light Chain(1656)
DIQLTQSPSFLSTSVGDRVTITCRASQGISNYLAWYQQKPGTAPKLLIYSASTLQSGVPSRFSGSGSGTEFTLTISSLQPEDFATYYCQHLKTSLVTFGQGTRLEM

SEQ ID NO: 8 - protein, 131 aa, Homo sapiens
  SITE (1)..(131): Variable Region of Heavy Chain(1656)
EVQLVESGGGLVQPGGSLRLSCSASGFTDSRFTFRNYWMHWVRQAPGKGLVWVSRINGDGGGTVYSDSVKGRFTISRDNARNIVYLQMDSLRAEDTAVYYCVREFGLFDSGTWYSGGFDYWGQGTLVTVSS

SEQ ID NO: 9 - protein, 6 aa, Homo sapiens
  SITE (1)..(6): CDR1 of Light Chain(1640)
QSIGKY

SEQ ID NO: 10 - protein, 3 aa, Homo sapiens
  SITE (1)..(3): CDR2 of Light Chain(1640)
AAS

SEQ ID NO: 11 - protein, 11 aa, Homo sapiens
  SITE (1)..(11): CDR3 of Light Chain(1640)
QQSFTAPQKYT

SEQ ID NO: 12 - protein, 10 aa, Homo sapiens
  SITE (1)..(10): CDR1 of Heavy Chain(1640)
GFTIPFRKYW

SEQ ID NO: 13 - protein, 8 aa, Homo sapiens
  SITE (1)..(8): CDR2 of Heavy Chain(1640)
IIGDGSST

SEQ ID NO: 14 - protein, 19 aa, Homo sapiens
  SITE (1)..(19): CDR3 of Heavy Chain(1640)
ARDLGERTGDNWHSLGPDY

SEQ ID NO: 15 - protein, 109 aa, Homo sapiens
  SITE (1)..(109): Variable Region of Light Chain(1640)
DIQMTQSPSSLSASVGDRVTITCRASQSIGKYLNWYQQKPGKAPKLLIYAASNLQSGVPSRFGGSGSGTDFTLTISSLQPEDCATYYCQQSFTAPQKYTFGQGTKLEIK

SEQ ID NO: 16 - protein, 128 aa, Homo sapiens
  SITE (1)..(128): Variable Region of Heavy Chain(1640)
EVQLVESGGGLVQPGGSLRLSCAASGFTIPFRKYWLHWVRQAPGKGLVWVSLIIGDGSSTNYADSVKGRFTISRDNDKNTLYLQMNSLRVEDTAVYYCARDLGERTGDNWHSLGPDYWGQGTLVTVSS

SEQ ID NO: 17 - protein, 7 aa, Homo sapiens
  SITE (1)..(7): CDR1 of Light Chain(2459)
QSVGFNY

SEQ ID NO: 18 - protein, 3 aa, Homo sapiens
  SITE (1)..(3): CDR2 of Light Chain(2459)
GAS

SEQ ID NO: 19 - protein, 9 aa, Homo sapiens
  SITE (1)..(9): CDR3 of Light Chain(2459)
QQYGNIPRT

SEQ ID NO: 20 - protein, 8 aa, Homo sapiens
  SITE (1)..(8): CDR1 of Heavy Chain(2459)
GFSFSTSG

SEQ ID NO: 21 - protein, 8 aa, Homo sapiens
  SITE (1)..(8): CDR2 of Heavy Chain(2459)
ITGDGANT

SEQ ID NO: 22 - protein, 17 aa, Homo sapiens
  SITE (1)..(17): CDR3 of Heavy Chain(2459)
ARAPAQHNTIWYGPDDY

SEQ ID NO: 23 - protein, 107 aa, Homo sapiens
  SITE (1)..(107): Variable Region of Light Chain(2459)
EIVLTQSPGTLSLSPGERATLSCGASQSVGFNYLAWYQQKPGQAPRLLIYGASNRATDIPDRFSGSGSGTDFTLTISRLEPEDFAVYYCQQYGNIPRTFGQGTKVEI

SEQ ID NO: 24 - protein, 124 aa, Homo sapiens
  SITE (1)..(124): Variable Region of Heavy Chain(2459)
QVQLVESGGGLVQPGGSLRLSCSTSGFSFSTSGMHWVRQAPGKGLEYVAAITGDGANTYYAKSVTGRFTVSRDNSKTTLYLQMSSLRTEDTAVYYCARAPAQHNTIWYGPDDYWGQGTLVTVSS

SEQ ID NO: 25 - DNA, 318 bp, Homo sapiens
  V_region (1)..(318): Variable Region of Light Chain(1656)
gacatccagctgacccagagccccagcttcctgagcaccagcgtgggcgaccgggtgaccatcacctgccgggccagccagggcatcagcaactacctggcctggtatcagcagaagcccggcaccgcccccaagctgctgatctacagcgccagcaccctgcagagcggcgtgcccagccggtttagcggcagcggctccggcaccgagttcaccctgaccatcagcagcctgcagcccgaggacttcgccacctactactgccagcacctgaaaaccagcctggtgaccttcggccagggcacccggctggaaatg

SEQ ID NO: 26 - DNA, 393 bp, Homo sapiens
  V_region (1)..(393): Variable Region of Heavy Chain(1656)
gaggtgcagctggtcgagtctggcggcggactggtgcagcctggcggcagcctgagactgagctgcagcgccagcggcttcaccgacagccggttcaccttccggaactactggatgcactgggtgcggcaggctccaggcaagggactggtgtgggtgtcccggatcaacggcgacggcggagggaccgtgtacagcgacagcgtgaagggcaggttcaccatcagccgggacaacgcccggaacatcgtgtacctgcagatggacagcctgcgggccgaggacaccgccgtgtactactgcgtgcgggagttcggcctgttcgacagcggcacctggtacagcggcggcttcgactactggggccagggcaccctggtgaccgtctcgagt

SEQ ID NO: 27 - DNA, 327 bp, Homo sapiens
  V_region (1)..(327): Variable Region of Light Chain(1640)
gacatccagatgacccagagccccagcagcctgagcgccagcgtgggcgaccgggtgaccatcacctgccgggccagccagagcatcggcaagtacctgaactggtatcagcagaagcccggcaaggcccccaagctgctgatctacgccgccagcaacctgcagagcggcgtgcccagcaggtttggcggcagcggctccggcaccgacttcaccctgaccatcagcagcctgcagcccgaggactgcgccacctactactgccagcagagcttcaccgcccctcagaagtacaccttcggccagggcaccaagctggaaatcaag

SEQ ID NO: 28 - DNA, 384 bp, Homo sapiens
  V_region (1)..(384): Variable Region of Heavy Chain(1640)
gaggtgcagctggtcgagtctggcggcggactggtgcagcctgggggaagcctgagactgagctgcgccgccagcggcttcaccatccccttccggaagtactggctgcactgggtgcggcaggccccaggcaagggactggtgtgggtgtccctgatcatcggcgacggcagcagcaccaactacgccgacagcgtgaagggccggttcaccatcagccgggacaacgacaagaacaccctgtacctgcagatgaacagcctgcgggtggaggacaccgccgtgtactactgcgccagggacctgggcgagcggaccggcgacaactggcacagcctgggccccgactattggggacagggcaccctggtgaccgtctcgagt

SEQ ID NO: 29 - DNA, 321 bp, Homo sapiens
  V_region (1)..(321): Variable Region of Light Chain(2459)
gagatcgtgctgacccagagccccggcaccctgagcctgagccctggcgagcgggccaccctgagctgtggcgccagccagagcgtgggcttcaactacctggcctggtatcagcagaagcccggccaggcccccaggctgctgatctacggcgccagcaaccgggccaccgacatccccgaccggttcagcggcagcggctccggcaccgacttcaccctgaccatcagccggctggaacccgaggacttcgccgtgtactactgccagcagtacggcaacatcccccggaccttcggccagggcaccaaggtggagatc

SEQ ID NO: 30 - DNA, 372 bp, Homo sapiens
  V_region (1)..(372): Variable Region of Heavy Chain(2459)
caggtgcagctggtcgagtctggcggcggactggtgcagcctggcggcagcctgagactgagctgcagcaccagcggcttcagcttcagcacctctggcatgcactgggtgcgccaggcccctggcaagggactggaatacgtggccgccatcaccggcgacggcgccaacacctactacgccaagagcgtgaccggcaggttcaccgtgtccagggacaacagcaagaccaccctgtacctgcagatgagcagcctgcggaccgaggacaccgccgtgtactactgcgccagggccccagcccagcacaacaccatttggtacggccccgacgactactggggacagggcaccctggtgaccgtctcgagt

SEQ ID NO: 31 - DNA, 38 bp, Artificial Sequence
  Synthetic polynucleotide
atatatatgcggccgcttattaacactctcccctgttg

SEQ ID NO: 32 - DNA, 20 bp, Artificial Sequence
  Synthetic polynucleotide
gacsgatgggcccttggtgg

SEQ ID NO: 33 - DNA, 20 bp, Artificial Sequence
  Synthetic polynucleotide
gagtggctcctgggggaaga

SEQ ID NO: 34 - DNA, 36 bp, Artificial Sequence
  Synthetic polynucleotide
tattcccatggcgcgcccagrtgcagctggtgcart

SEQ ID NO: 35 - DNA, 36 bp, Artificial Sequence
  Synthetic polynucleotide
tattcccatggcgcgccsaggtccagctggtrcagt

SEQ ID NO: 36 - DNA, 36 bp, Artificial Sequence
  Synthetic polynucleotide
tattcccatggcgcgcccagrtcaccttgaaggagt

SEQ ID NO: 37 - DNA, 35 bp, Artificial Sequence
  Synthetic polynucleotide
tattcccatggcgcgccsaggtgcagctggtggag

SEQ ID NO: 38 - DNA, 36 bp, Artificial Sequence
  Synthetic polynucleotide
tattcccatggcgcgcccaggtgcagctacagcagt

SEQ ID NO: 39 - DNA, 36 bp, Artificial Sequence
  Synthetic polynucleotide
tattcccatggcgcgcccagstgcagctgcaggagt

SEQ ID NO: 40 - DNA, 36 bp, Artificial Sequence
  Synthetic polynucleotide
tattcccatggcgcgccgargtgcagctggtgcagt

SEQ ID NO: 41 - DNA, 37 bp, Artificial Sequence
  Synthetic polynucleotide
tattcccatggcgcgcccaggtacagctgcagcagtc

SEQ ID NO: 42 - DNA, 45 bp, Artificial Sequence
  Synthetic polynucleotide
ggcgcgccatgggaatagctagccgacatccagwtgacccagtct

SEQ ID NO: 43 - DNA, 45 bp, Artificial Sequence
  Synthetic polynucleotide
ggcgcgccatgggaatagctagccgatgttgtgatgactcagtct

SEQ ID NO: 44 - DNA, 45 bp, Artificial Sequence
  Synthetic polynucleotide
ggcgcgccatgggaatagctagccgaaattgtgwtgacrcagtct

SEQ ID NO: 45 - DNA, 45 bp, Artificial Sequence
  Synthetic polynucleotide
ggcgcgccatgggaatagctagccgatattgtgatgacccacact

SEQ ID NO: 46 - DNA, 43 bp, Artificial Sequence
  Synthetic polynucleotide
ggcgcgccatgggaatagctagccgaaacgacactcacgcagt

SEQ ID NO: 47 - DNA, 45 bp, Artificial Sequence
  Synthetic polynucleotide
ggcgcgccatgggaatagctagccgaaattgtgctgactcagtct

SEQ ID NO: 48 - DNA, 51 bp, Artificial Sequence
  Synthetic polynucleotide
accgcctccaccggcggccgcttattaacactctcccctgttgaagctctt

SEQ ID NO: 49 - DNA, 30 bp, Artificial Sequence
  Synthetic polynucleotide
ggaggcgctcgagacggtgaccagggtgcc

SEQ ID NO: 50 - DNA, 30 bp, Artificial Sequence
  Synthetic polynucleotide
ggaggcgctcgagacggtgaccattgtccc

SEQ ID NO: 51 - DNA, 30 bp, Artificial Sequence
  Synthetic polynucleotide
ggaggcgctcgagacggtgaccagggttcc

SEQ ID NO: 52 - DNA, 30 bp, Artificial Sequence
  Synthetic polynucleotide
ggaggcgctcgagacggtgaccgtggtccc

(19) United States
(12) Patent Application Publication
(10) Pub. No.: US 2013/0004499 A1
(43) Pub. Date: Jan. 3, 2013

(54) ANTIBODY AGAINST SEROTYPE E LIPOPOLYSACCHARIDE OF PSEUDOMONAS AERUGINOSA

(75) Inventors: Jiro Tanaka, Yokohama-shi (JP); Peter Sejer Andersen, Vanlose (DK); Takafumi Okutomi, Yokohama-shi (JP); Tsuneyoshi Inaba, Yokohama-shi (JP); Keiko Otsuka, Yokohama-shi (JP); Hirotomo Akabane, Yokohama-shi (JP); Yukari Hoshina, Yokohama-shi (JP); Jun Saito, Yokohama-shi (JP); Hiroshi Nagaso, Yokohama-shi (JP); Masashi Kumagai, Yokohama-shi (JP); Yasuyo Hagiwara, Yokohama-shi (JP)

(73) Assignees: SYMPHOGEN A/S, Lyngby (DK); Meiji Seika Pharma Co., Ltd., Tokyo (JP)

(21) Appl. No.: 13/579,757

(22) PCT Filed: Feb. 18, 2011
(86) PCT No.: PCT/JP2011/054223

§ 371 Date: Sep. 21, 2012

(30) Foreign Application Priority Data
Feb. 18, 2010 (JP) 2010-033429

(51) Int. Cl. C07K 16/12; C12N 5/12; A61P 17/02; A61P 31/04; A61P 11/00; C12N 15/13; A61K 39/395

(52) U.S. Cl. 424/137.1; 536/23.53; 435/329; 530/387.5; 530/387.3

(57) ABSTRACT

Provided is a novel antibody having an excellent antibacterial activity against P. aeruginosa. By using plasmablasts obtained from cystic fibrosis patients with chronic P. aeruginosa pulmonary infection as starting materials, antibodies which bind to LPS of a P. aeruginosa strain of serotype E and which have excellent antibacterial activities in vitro and in vivo were successfully obtained.

### TECHNICAL FIELD

The present invention relates to an antibody against serotype E lipopolysaccharide of P. aeruginosa and applications thereof. More specifically, the present invention relates to an antibody which specifically binds to serotype E lipopolysaccharide of a P. aeruginosa strain, and a pharmaceutical composition, a diagnostic agent for a P. aeruginosa infection, and a P. aeruginosa detection kit, each including any of the antibodies.

### BACKGROUND ART

P. aeruginosa (Pseudomonas aeruginosa) is a gram-negative aerobic bacillus widely and generally distributed in natural environments such as soil and water. P. aeruginosa is an avirulent bacterium which normally is not pathogenic to healthy subjects, who have a moderate antibody titer and a sufficient immune function against P. aeruginosa. However, once debilitated patients are infected with P. aeruginosa, P. aeruginosa may cause severe symptoms, which may lead to the death of the patients. For this reason, P. aeruginosa has attracted attention as a major causative bacterium of nosocomial infections and opportunistic infections, and hence the prevention and treatment of P. aeruginosa infections have been important issues in the medical field.

For the prevention or treatment of P. aeruginosa infections, antibiotics or synthetic antibacterial agents have mainly been used. However, P. aeruginosa develops resistance to such medicines, and hence such medicines do not provide a sufficient therapeutic effect in many cases. Particularly, treatment of infections with multi-drug resistant P. aeruginosa (MDRP) using antibiotics or the like is difficult, and has limitation. For this reason, as an alternative method thereto, treatment using an immunoglobulin preparation has been conducted.

Meanwhile, the prevention or treatment of a P. aeruginosa infection using an antibody against P. aeruginosa has been examined. For example, antibodies each of which specifically binds to a P. aeruginosa strain of a specific serotype have been developed (Patent Literatures 1 to 5, and Non-Patent Literatures 1 and 2). However, the antibodies against P. aeruginosa developed so far do not provide a sufficient effect in prevention or treatment of a P. aeruginosa infection.

### CITATION LIST

### Patent Literature

[PTL 1] Japanese Unexamined Patent Application Publication No. Hei 6-178688
[PTL 2] Japanese Unexamined Patent Application Publication No. Hei 6-178689
[PTL 3] Japanese Unexamined Patent Application Publication No. Hei 7-327677
[PTL 4] International Publication No. WO2004/101622
[PTL 5] International Publication No. WO2006/084758

### Non Patent Literature

[NPL 1] The Journal of Infectious Diseases, 152, 6, 1985, 1290-1299.
[NPL 2] Journal of General Microbiology, 133, 1987, 3581-3590.

### SUMMARY OF INVENTION

### Technical Problem

The present invention has been made in view of the above-described circumstances, and an object of the present invention is to provide a novel antibody which has an excellent antibacterial activity against P. aeruginosa. One main object of the present invention is to provide an antibody which specifically binds to serotype E lipopolysaccharide of a P. aeruginosa strain.

### Solution to Problem

To achieve the above-described object, the present inventors employed the following approach. First, blood samples were collected from cystic fibrosis patients with chronic P. aeruginosa pulmonary infection and healthy volunteers. Donor specimens having a high proportion of plasmablasts which were specific to lipopolysaccharide (hereinafter sometimes simply referred to as “LPS”) were identified by: (1) FACS analysis which determined the amounts of plasmablasts and plasmacytes in the circulating blood; (2) ELISPOT analysis which determined the amount of cells, in the circulating blood, produceing antibodies secific to a specific LPS antigen; and (3) ELISA analysis which determined the presence or absence of immunoglobulins specific to a specific LPS antigen. Next, antibodies which recognized LPS were prepared from the donor specimens thus identified.

Specifically, viable plasmablasts were selected by staining CD19, CD38, A light chain, and dead cells. On the selected plasmablasts, the pairing of DNA sequences coding a heavy chain variable region (VH) and a light chain variable region (VL) which were originated from the same B cell by two-stage PCR involving multiplex overlap-extension RT-PCR and subsequent nested PCR (FIG. 1). Next, amplified DNA was inserted into a screening vector, and then transformed into Escherichia coli. A repertoire of the amplified vector was purified from the Escherichia coli. The obtained antibody library was expressed in animal culture cells. Clones coding antibodies which bound to purified LPS molecules were screened by ELISA, and LPS-specific clones were selected. Then, the base sequences of the selected clones were determined. Thereafter, antibodies coded by the thus obtained clones were examined for their various activities, their serotype specificity, and epitopes.

As a result, it is found out that identified antibodies bind to serotype E LPS of P. aeruginosa, and have excellent antibacterial activities in vitro and in vivo.

Specifically, the present invention relates to antibodies which bind to serotype E LPS of P. aeruginosa, show an excellent antibacterial activity. The present invention also relates to applications of the antibodies. More specifically, the present invention provides

[1] An antibody which recognizes B-band LPS of lipopolysaccharides of P. aeruginosa, and which substantially binds to a surface of a P. aeruginosa strain of serotype E, but does not substantially binds to any one of surfaces of P. aeruginosa strains of serotype A, B, C, D, F, G, H, I and M.
[2] The antibody according to clause 1, which has an opsonic activity against a P. aeruginosa strain of serotype E.
[3] The antibody according to clause 2, wherein an EC50 of an opsonic activity against a P. aeruginosa strain identified by ATCC 29260 is 1 μg/ml or less.
[4] The antibody according to any one of clauses 1 to 3, which has an agglutination activity against a P. aeruginosa strain of serotype E.
[5] The antibody according to clause 4, wherein an agglutination titer per amount (μg) of IgG against a P. aeruginosa strain identified by ATCC 29260 is 100 or more.
[6] The antibody according to any one of clauses 1 to 5, which has an antibacterial effect against a systemic infection with a P. aeruginosa strain of serotype E.
[7] The antibody according to clause 6, wherein an ED50 of an antibacterial effect on a neutropenic mouse model of systemic infection with a P. aeruginosa strain identified by ATCC 29260 is not more than 1/30 of that of Venilon.
[8] The antibody according to any one of clauses 1 to 7, which has an antibacterial effect against a pulmonary infection with a P. aeruginosa strain of serotype E.
[9] The antibody according to clause 8, wherein an antibacterial effect on a mouse model of pulmonary infection with a P. aeruginosa strain identified by ATCC 29260 has at least one property selected from the following group:

(a) upon administration of the antibody to a mouse immediately after the inoculation with a P. aeruginosa strain identified by ATCC 29260 to the mouse, an ED50 of the antibacterial effect on the mouse is not more than 1/500 of that of Venilon; and

(b) upon administration of the antibody to a mouse 8 hours after the inoculation with a P. aeruginosa strain identified by ATCC 29260 to the mouse, an ED50 of the antibacterial effect on the mouse is not more than 1/3000 of that of Venilon.

[10] The antibody according to any one of clauses 1 to 9, which has an antibacterial effect against a burn wound infection with a P. aeruginosa strain of serotype E.
[11] The antibody according to clause 10, wherein an antibacterial effect on a mouse model of burn wound infection with a P. aeruginosa strain identified by ATCC 29260 has at least one property selected from the following group:

(a) upon administration of the antibody to a mouse immediately after the inoculation with a P. aeruginosa strain identified by ATCC 29260 to the mouse, an ED50 of the antibacterial effect on the mouse is not more than 1/1500 of that of Venilon; and

(b) upon administration of the antibody to a mouse 25 hours after the inoculation with a P. aeruginosa strain identified by ATCC 29260 to the mouse, an ED50 of the antibacterial effect on the mouse is not more than 1/2000 of that of Venilon.

[12] The antibody which has any one of the following features (a) and (b):

(a) comprising
a light chain variable region including amino acid sequences described in SEQ ID NOs: 1 to 3 or the amino acid sequences described in SEQ ID NOs: 1 to 3 in at least one of which one or more amino acids are substituted, deleted, added, and/or inserted, and
a heavy chain variable region including amino acid sequences described in SEQ ID NOs: 4 to 6 or the amino acid sequences described in SEQ ID NOs: 4 to 6 in at least one of which one or more amino acids are substituted, deleted, added, and/or inserted; and

(b) comprising
a light chain variable region including amino acid sequences described in SEQ ID NOs: 9 to 11 or the amino acid sequences described in SEQ ID NOs: 9 to 11 in at least one of which one or more amino acids are substituted, deleted, added, and/or inserted, and
a heavy chain variable region including amino acid sequences described in SEQ ID NOs: 12 to 14 or the amino acid sequences described in SEQ ID NOs: 12 to 14 in at least one of which one or more amino acids are substituted, deleted, added, and/or inserted.
   [13] The antibody which has any one of the following features (a) and (b):

(a) comprising
a light chain variable region including an amino acid sequence described in SEQ ID NO: 7 or the amino acid sequence described in SEQ ID NO: 7 in which one or more amino acids are substituted, deleted, added, and/or inserted, and
a heavy chain variable region including an amino acid sequence described in SEQ ID NO: 8 or the amino acid sequence described in SEQ ID NO: 8 in which one or more amino acids are substituted, deleted, added, and/or inserted; and

(b) comprising
a light chain variable region including an amino acid sequence described in SEQ ID NO: 15 or the amino acid sequence described in SEQ ID NO: 15 in which one or more amino acids are substituted, deleted, added, and/or inserted, and
a heavy chain variable region including an amino acid sequence described in SEQ ID NO: 16 or the amino acid sequence described in SEQ ID NO: 16 in which one or more amino acids are substituted, deleted, added, and/or inserted.
   [14] A peptide comprising a light chain or a light chain variable region of the antibody, the peptide having any one of the following features (a) and (b):

(a) comprising amino acid sequences described in SEQ ID NOs: 1 to 3 or the amino acid sequences described in SEQ ID NOs: 1 to 3 in at least one of which one or more amino acids are substituted, deleted, added, and/or inserted; and

(b) comprising amino acid sequences described in SEQ ID NOs: 9 to 11 or the amino acid sequences described in SEQ ID NOs: 9 to 11 in at least one of which one or more amino acids are substituted, deleted, added, and/or inserted.

[15] A peptide comprising a light chain or a light chain variable region of the antibody, the peptide having any one of the following features (a) and (b):

(a) comprising an amino acid sequence described in SEQ ID NO: 7 or the amino acid sequence described in SEQ ID NO: 7 in which one or more amino acids are substituted, deleted, added, and/or inserted; and

(b) comprising an amino acid sequence described in SEQ ID NO: 15 or the amino acid sequence described in SEQ ID NO: 15 in which one or more amino acids are substituted, deleted, added, and/or inserted.

[16] A peptide comprising a heavy chain or a heavy chain variable region of the antibody, which has any one of the following features (a) and (b):

(a) comprising amino acid sequences described in SEQ ID NOs: 4 to 6 or the amino acid sequences described in SEQ ID NOs: 4 to 6 in at least one of which one or more amino acids are substituted, deleted, added, and/or inserted; and

(b) comprising amino acid sequences described in SEQ ID NOs: 12 to 14 or the amino acid sequences described in SEQ ID NOs: 12 to 14 in at least one of which one or more amino acids are substituted, deleted, added, and/or inserted.

[17] A peptide comprising a heavy chain or a heavy chain variable region of the antibody, which has any one of the following features (a) and (b):

(a) comprising an amino acid sequence described in SEQ ID NO: 8 or the amino acid sequence described in SEQ ID NO: 8 in which one or more amino acids are substituted, deleted, added, and/or inserted; and

(b) comprising an amino acid sequence described in SEQ ID NO: 16 or the amino acid sequence described in SEQ ID NO: 16 in which one or more amino acids are substituted, deleted, added, and/or inserted.

[18] An antibody which binds to an epitope, in B-band LPS of lipopolysaccharides of a P. aeruginosa strain of serotype E, of an antibody described in any one of the following (a) and (b):

(a) an antibody comprising a light chain variable region including an amino acid sequence described in SEQ ID NO: 7, and a heavy chain variable region including an amino acid sequence described in SEQ ID NO: 8; and

(b) an antibody comprising a light chain variable region including an amino acid sequence described in SEQ ID NO: 15 and a heavy chain variable region including an amino acid sequence described in SEQ ID NO: 16.

[19] A DNA which codes the antibody or the peptide according to any one of clauses 1 to 18.
[20] A hybridoma which produces the antibody according to any one of clauses 1 to 13, and 18.
[21] A pharmaceutical composition for a disease associated with P. aeruginosa, the pharmaceutical composition comprising:

the antibody according any one of clauses 1 to 13, and 18; and optionally

at least one pharmaceutically acceptable carrier and/or diluent.

[22] The pharmaceutical composition according to clause 21, wherein the disease associated with P. aeruginosa is a systemic infectious disease caused by a P. aeruginosa infection.
[23] The pharmaceutical composition according to clause 21, wherein the disease associated with P. aeruginosa is a pulmonary infectious disease caused by a P. aeruginosa infection.
[24] The pharmaceutical composition according to clause 21, wherein the disease associated with P. aeruginosa is a burn wound infectious disease caused by a P. aeruginosa infection.
[25] A diagnostic agent for detection of P. aeruginosa, the diagnostic agent comprising: the antibody according any one of clauses 1, 12, 13, and 18.
[26] A kit for detection of P. aeruginosa, the kit comprising: the antibody according any one of clauses 1, 12, 13, and 18.

### Advantageous Effects of Invention

The present invention provides an antibody which binds to serotype E LPS of P. aeruginosa, and which exhibits an excellent antibacterial activity. The antibody of the present invention can exhibit an excellent opsonic effect and an excellent antibacterial effect against a systemic infection, pulmonary infection, or a burn wound infection with P. aeruginosa. Moreover, since the antibody of the present invention is originated from cystic fibrosis patients with chronic P. aeruginosa pulmonary infection, an excellent effect against clinical P. aeruginosa strains can be expected. The antibody of the present invention can be prepared as a human antibody, and hence is highly safe. The use of an antibody of the present invention makes it possible to effectively treat or prevent infections, such as HAP/VAP, bacteremia, septicemia, and burn wound infection, which are caused by P. aeruginosa, including multi-drug resistant P. aeruginosa.

### BRIEF DESCRIPTION OF DRAWINGS

FIG. 1 is a diagram showing two-stage PCR performed to obtain DNA coding an antibody of the present invention.

FIG. 2 is a diagram showing an OO-VP-002 vector used for the pairing of sequences coding a heavy chain variable region (VH) and a light chain variable region (VL), which were originated from the same B cell.

FIG. 3 is a graph showing analysis results of an additive effect of the antibody “2459” with an antibody “1656” by SPR measurement.

### DESCRIPTION OF EMBODIMENTS

The present invention provides a novel antibody which binds to serotype E LPS of P. aeruginosa. An “antibody” in the present invention includes all classes and all subclasses of immunoglobulins. The “antibody” includes a polyclonal antibody and a monoclonal antibody, and also includes the form of a functional fragment of an antibody. A “polyclonal antibody” refers to an antibody preparation comprising different kinds of antibodies against different epitopes. Meanwhile, a “monoclonal antibody” means an antibody (including antibody fragments) obtained from a substantially homogeneous population of antibodies. In contrast to the polyclonal antibody, the monoclonal antibody recognizes a single determinant on an antigen. The polyclonal antibody in the present invention also includes a combination of multiple monoclonal antibodies capable of recognizing multiple epitopes on an antigen. The antibody of the present invention is an isolated antibody, that is, an antibody which is separated and/or recovered from components in a natural environment.

A “lipopolysaccharide (LPS)” to which the antibody of the present invention binds is a constituent of an outer membrane of a cell wall of a Gram-negative bacterium, and is a substance formed of a lipid and a polysaccharide (a glycolipid). The carbohydrate chain is formed of a moiety called a core polysaccharide (or a core oligosaccharide), and a moiety called an O antigen (an O side chain polysaccharide). “A-band LPS” is a LPS whose polysaccharide forming the O antigen has the following structure. Specifically, in the structure, units each consisting of “3)-α-D-Rha-(1→2)-α-D-Rha-(1→3)-α-D-Rha-(1” are repeated. In these units, the D-rhamnose is linked by α-1,2 and α-1,3 bonds. The structural formula thereof is shown below; however, the branching mode of D-rhamnose linked by α-1,2-bonds and D-rhamnose linked by α-1,3-bonds is not limited to that shown below.

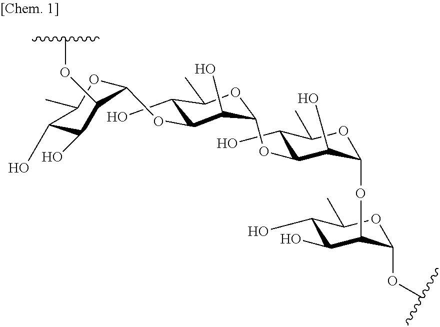

Meanwhile, “B-band LPS” is serotype-specific LPS having a structure in which units each consisting of bonds of two to five sugars in polysaccharide forming the O antigen are repeated. As will be described below, the structure of the repeating units in the B-band LPS of P. aeruginosa strains are different from one another, depending on their serotypes (refer to Microbiol. Mol. Biol. Rev. 63 523-553 (1999)).

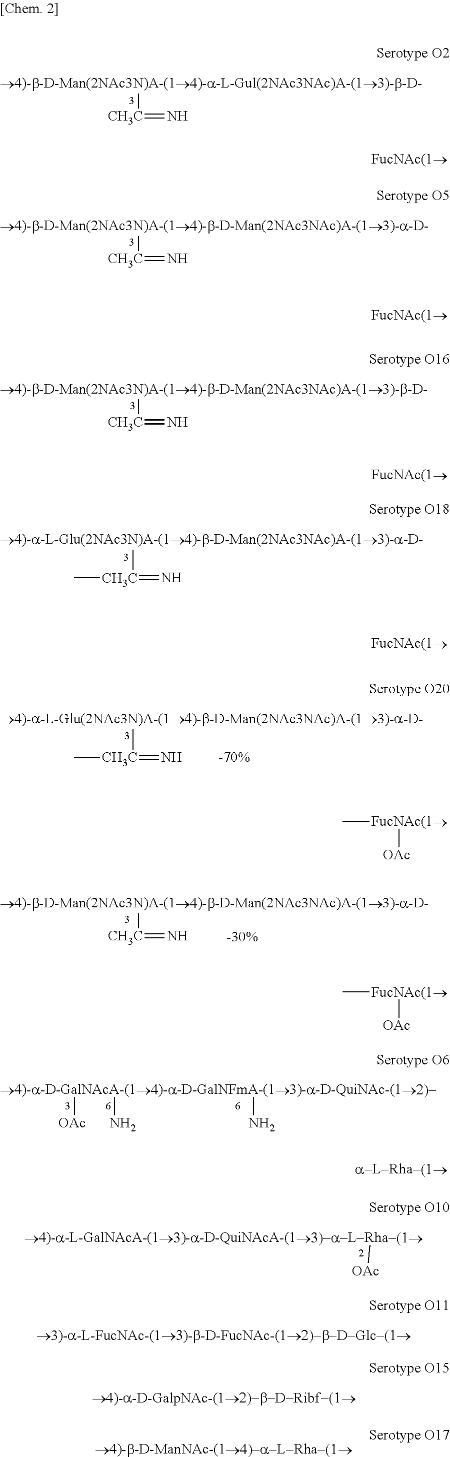

A “serotype” in the present invention means any known serotype of P. aeruginosa. Table 1 shows the correspondence of groups according to the serotyping committee sponsored by Japan P. aeruginosa Society, with types according to IATS (International Antigenic Typing System), both being currently used for P. aeruginosa strains of different serotypes. The serotype of a P. aeruginosa strain can be determined by using a commercially-available immune serum for grouping of P. aeruginosa.

TABLE 1

| JPAS | IATS |
|---|---|
| I | O1 |
| B | O2 |
| A | O3 |
| F | O4 |
| B | O5 |
| C | O7 |
| G | O6 |
| C | O8 |
| D | O9 |
| H | O10 |
| E | O11 |
| L | O12 |
| K | O13 |
| K | O14 |
| J | O15 |
| B | O16 |
| N | O17 |
| — | O18 |
| — | O19 |
| B | O20 |

JPAS: Japan P. aeruginosa society
IATS: International Antigenic Typing System
Reference Document: Microbiology 17 273-304 (1990)

Out of the antibodies identified in the present invention, an antibody “1656” and an antibody “1640” exhibited an excellent specificity to a P. aeruginosa strain of serotype E. Accordingly, another embodiment of the antibody of the present invention is an antibody which specifically binds to lipopolysaccharide of a P. aeruginosa strain of serotype E (hereinafter referred to as an “anti-serotype E LPS antibody”). The anti-serotype E LPS antibody of the present invention is preferably an antibody which recognizes lipopolysaccharide of P. aeruginosa, and which substantially binds to a surface of a P. aeruginosa strain of serotype E, but does not substantially bind to any one of surfaces of P. aeruginosa strains of serotype A, C, D, F, G, H, I, and M. For the anti-serotype E LPS antibody of the present invention, the phrase “substantially binds to” means, for example, that an absorbance, which is indicative of binding capability, is 0.25 or more, when detected by the whole-cell ELISA method described in the examples of the present application. Meanwhile, the phrase “does not substantially bind to” means, for example, that an absorbance, which is indicative of binding capability, is less than 0.25, when detected by the whole-cell ELISA method described in the examples of the present application.

Examples of P. aeruginosa strains of serotype A include those with ATCC accession Nos. 27577 and 33350. Examples of P. aeruginosa strains of serotype B include those with 27578, 33349, BAA-47, 33352, 33363 and 43732. Examples of P. aeruginosa strains of serotype C include those with 33353, 27317 and 33355. Examples of P. aeruginosa strains of serotype D include those with 27580 and 33356. Examples of P. aeruginosa strains of serotype E include those with 29260 and 33358. Examples of P. aeruginosa strains of serotype F include those with 27582 and 33351. Examples of P. aeruginosa strains of serotype G include those with 27584 and 33354. Examples of P. aeruginosa strains of serotype H include those with 27316 and 33357. Examples of P. aeruginosa strains of serotype I include those with 27586 and 33348. An example of P. aeruginosa strains of serotype J is one with 33362. Examples of P. aeruginosa strains of serotype K include those with 33360 and 33361. An example of P. aeruginosa strains of serotype L is one with 33359. An example of P. aeruginosa strains of serotype M is one with 21636. An example of P. aeruginosa strains of serotype N is one with 33364.

Examples of P. aeruginosa strains of the other serotype (O18 type and O19 type) include those with 43390 and 43731. Examples of P. aeruginosa strains of serotype E include multi-drug resistant P. aeruginosa (MDRP) strains of serotype E/O11(MSC 06120, MSC 17660, MSC 17661, MSC 17662, MSC 17667, MSC 17671, MSC 17693, MSC 17727, MSC 17728, or the like) possessed by MEIJI SEIKA KAISHA, LTD. Note that Multidrug resistance in the present invention is defined as resistance to at least three of the following agents according to CLSI breakpoints: imipenem (≧16 μg/ml), ceftazidime (≧32 μg/ml), tobramycin (≧16 μg/ml), ciprofloxacin (4 μg/ml). (Reference: National Surveillance of Antimicrobial Resistance in Pseudomonas aeruginosa Isolates Obtained from Intensive Care Unit Patients from 1993 to 2002, Marilee D. Obritsch, Douglas N. Fish, Robert MacLaren, and Rose Jung, ANTIMICROBIAL AGENTS AND CHEMOTHERAPY, 48. 12. 2004, 4606-4610)

The anti-serotype E LPS antibody of the present invention is preferably an antibody which substantially binds to only P. aeruginosa of serotype E, but which does not substantially binds to any one of P. aeruginosa strains of the other serotypes, out of the P. aeruginosa strains identified by the ATCC accession numbers shown as examples above. Moreover, the anti-serotype E LPS antibody of the present invention is preferably an antibody which substantially binds to MDRP of serotype E/O11 possessed by MEIJI SEIKA KAISHA, LTD. More preferably, the anti-serotype E LPS antibody of the present invention is an antibody which substantially binds to all the P. aeruginosa strains of serotype E, but which does not substantially binds to any one of P. aeruginosa strains of the other serotypes, out of the P. aeruginosa strains identified by the ATCC accession numbers shown as examples above.

According to a preferred embodiment, the anti-serotype E LPS antibody of the present invention has an opsonic activity against P. aeruginosa. The anti-serotype E LPS antibody of the present invention can have an opsonic activity against a P. aeruginosa strain of serotype E, as a reflection of the binding activity to a P. aeruginosa strain of serotype E. In particular, the antibody “1656” and the antibody “1640” of the present invention each exhibited a high opsonic activity against a P. aeruginosa strain of serotype E. Particularly notably, when the opsonic activities of the antibody “1656” and the antibody “1640” of the present invention were evaluated by using the P. aeruginosa strain of serotype E (ATCC 29260) and by employing the detection method using, as an index, a fluorescence intensity of human polymorphonuclear leukocytes incorporating FITC-labeled P. aeruginosa, as described in the examples of the present application, the EC50s of “the antibody “1656” and the antibody “1640” were 0.11 and 0.64 μg/ml, respectively. The anti-serotype E LPS antibody of the present invention preferably has such an excellent opsonic activity, and is, for example, an antibody of which EC50 of opsonic activity against the P. aeruginosa strain of serotype E (ATCC 29260) is 1 μg/ml or less (for example, 0.8 μg/ml or less, 0.6 μg/ml or less, 0.4 μg/ml or less, or 0.3 μg/ml or less, 0.2 μg/ml or less).

Moreover, when the opsonic activity of the anti-serotype E LPS antibody of the present invention is evaluated by using the P. aeruginosa strain of serotype E (ATCC 29260) and by employing the detection method using, as an index, a fluorescence intensity of human polymorphonuclear leukocytes incorporating FITC-labeled P. aeruginosa, as described in the examples of the present application, the mean fluorescence intensity (MFI) value of the anti-serotype E LPS antibody at 30 μg/ml is preferably not less than 0.5 times (for example, not less than 0.8 times, not less than 1 time or not less than 1.2 times) the mean fluorescence intensity (MFI) value of Venilon at 1000 μg/ml.

According to another preferred embodiment, the anti-serotype E LPS antibody of the present invention has an agglutination activity against P. aeruginosa. The antibody “1656” of the present invention showed an excellent agglutination titer per amount (μg) of IgG 190, when the P. aeruginosa strain of serotype E (ATCC 29260) was used. Because of having such an excellent agglutination activity, the anti-serotype E LPS antibody of the present invention used as a medicine can induce an efficient opsonic activity even in a low dose, and hence an effect of infection prevention can be anticipated. The anti-serotype E LPS antibody of the present invention preferably has an agglutination titer per amount (μg) of IgG of 100 or more (for example, 150 or more, 170 or more or 190 or more), when the P. aeruginosa strain of serotype E (ATCC 29260) was used.

According to another preferred embodiment, the anti-serotype E LPS antibody of the present invention has an antibacterial effect against a systemic infection, a pulmonary infection, and a burn wound infection with P. aeruginosa. The antibody “1656” and the antibody “1640” of the present invention exhibited an antibacterial activity against a pulmonary infection with a P. aeruginosa strain of serotype E. Surprisingly, when a mouse model to which the antibody administered immediately after a pulmonary infection with a P. aeruginosa strain of serotype E (ATCC 29260) was used and comparison was made by using Venilon as a control, the ED50 value of antibacterial effect of each of the antibody “1656” and the antibody “1640” was 1/500 or less of the ED50 value of Venilon. In particular, the antibody “1656” exhibited such an excellent effect that the ED50 thereof was 1/1000 or less of that of Venilon. Accordingly, the ED50 value of the anti-serotype E LPS antibody of the present invention is preferably 1/500 or less (for example, 1/600 or less or 1/800 or less or 1/1000 or less) of that of Venilon, when the pulmonary infection mouse model is used.

Moreover, when a mouse model to which the antibody administered 8 hours after a pulmonary infection with a P. aeruginosa strain of serotype E (ATCC 29260) was used and comparison was made by using Venilon as a control, the ED50 value of antibacterial effect of the antibody “1656” was 1/3000 or less of the ED50 value of Venilon. Accordingly, the ED50 value of the anti-serotype E LPS antibody of the present invention is preferably 1/3000 or less (for example, 1/4000 or less or 1/5000 or less) of that of Venilon, when the pulmonary infection mouse model is used.

Furthermore, when a mouse model to which the antibody administered immediately after a pulmonary infection with a P. aeruginosa strain of serotype E (MSC 06120) was used and comparison was made by using Venilon as a control, the ED50 value of antibacterial effect of the antibody “1656” was 1/500 or less of the ED50 value of Venilon. Accordingly, the ED50 value of the anti-serotype E LPS antibody of the present invention is preferably 1/500 or less (for example, 1/600 or less or 1/700 or less) of that of Venilon, when the pulmonary infection mouse model is used. Moreover, when a mouse model to which the antibody administered 8 hours after a pulmonary infection with a P. aeruginosa strain of serotype E (MSC 06120) was used and comparison was made by using Venilon as a control, the ED50 value of antibacterial effect of the antibody “1656” was 1/50 or less of the ED50 value of Venilon. Accordingly, the ED50 value of the anti-serotype E LPS antibody of the present invention is preferably 1/50 or less (for example, 1/60 or less or 1/70 or less) of that of Venilon, when the pulmonary infection mouse model is used.

The antibody “1656” and the antibody “1640” of the present invention further exhibited an antibacterial activity against a systemic infection with a P. aeruginosa strain of serotype E. Surprisingly, when a neutropenic mouse model of systemic infection with P. aeruginosa identified by the P. aeruginosa strain of serotype E (ATCC 29260) was used and comparison was made by using Venilon as a control, the ED50 value of antibacterial effect of each of these antibodies was so excellent that each of the ED50 values exhibited was 1/30 or less of the ED50 value of Venilon. Particularly, for a mouse model of systemic infection with a P. aeruginosa strain identified by ATCC 29260, the antibody “1656” exhibited such an excellent effect that the ED50 value of the antibody “1656” was 1/140 or less of the ED50 value of Venilon. Accordingly, when a neutropenic mouse model of systemic infection is used, the ED50 value of the anti-serotype E LPS antibody of the present invention is preferably 1/30 or less (for example, 1/40 or less, 1/70 or less, 1/100 or less, 1/130 or less or 1/140 or less) of that of Venilon. Moreover, when a neutropenic mouse model of systemic infection with P. aeruginosa identified by the P. aeruginosa strain of serotype E (MSC 06120) was used and comparison was made by using Venilon as a control, the ED50 value of antibacterial effect of the antibody “1656” was 1/120 or less of the ED50 value of Venilon. Accordingly, when a neutropenic mouse model of systemic infection is used, the ED50 value of the anti-serotype E LPS antibody of the present invention is preferably 1/120 or less (for example, 1/150 or less or 1/180 or less) of that of Venilon.

The antibody “1656” of the present invention further exhibited an antibacterial activity against a burn wound infection with a P. aeruginosa strain of serotype E. Surprisingly, when a mouse model to which the antibody administered immediately after a burn wound infection with a P. aeruginosa strain of serotype E (ATCC 29260) was used and comparison was made by using Venilon as a control, the ED50 value of antibacterial effect of the antibody “1656” was 1/1500 or less of the ED50 value of Venilon. Accordingly, the ED50 value of the anti-serotype E LPS antibody of the present invention is preferably 1/1500 or less (for example, 1/2000 or less or 1/2500 or less) of that of Venilon, when the burn wound infection mouse model is used. Moreover, when a mouse model to which the antibody administered 25 hours after a burn wound infection with a P. aeruginosa strain of serotype E (ATCC 29260) was used and comparison was made by using Venilon as a control, the ED50 value of antibacterial effect of the antibody “1656” was 1/2000 or less of the ED50 value of Venilon. Accordingly, the ED50 value of the anti-serotype E LPS antibody of the present invention is preferably 1/2000 or less (for example, 1/2500 or less or 1/3000 or less) of that of Venilon, when the burn wound infection mouse model is used.

The anti-serotype E LPS antibody of the present invention can have any one of the above-described activities alone, but preferably has multiple activities together.

Another preferred embodiment of the anti-serotype E LPS antibody of the present invention is an antibody comprising a light chain variable region including light chain CDRs 1 to 3 and a heavy chain variable region including heavy chain CDRs 1 to 3, of the antibody (1656 or 1640) identified in the present invention. Specific examples thereof include the following antibodies (i) and (ii):

(i) an antibody comprising a light chain variable region including light chain CDRs 1 to 3 (amino acid sequences described in SEQ ID NOs: 1 to 3) and a heavy chain variable region including heavy chain CDRs 1 to 3 (amino acid sequences described in SEQ ID NOs: 4 to 6), for example, an antibody in which a light chain variable region includes an amino acid sequence described in SEQ ID NO: 7 and a heavy chain variable region includes an amino acid sequence described in SEQ ID NO: 8: and

(ii) an antibody comprising a light chain variable region including light chain CDRs 1 to 3 (amino acid sequences described in SEQ ID NOs: 9 to 11) and a heavy chain variable region including heavy chain CDRs 1 to 3 (amino acid sequences described in SEQ ID NOs: 12 to 14), for example, an antibody in which a light chain variable region includes an amino acid sequence described in SEQ ID NO: 15 and a heavy chain variable region includes an amino acid sequence described in SEQ ID NO: 16.

The present invention also provides a peptide comprising any one of a light chain, a heavy chain and variable regions thereof of an antibody, the peptide including CDR identified in the antibody (1656 or 1640) of the present invention.

Examples of a peptide comprising any one of a light chain, a heavy chain and variable regions thereof, of an antibody, the peptide comprising CDR of the antibody 1656, include the following peptides (i) and (ii):

(i) a peptide comprising a light chain or a light chain variable region of the antibody of the present invention, the peptide comprising the amino acid sequences described in SEQ ID NOs: 1 to 3, for example, a peptide comprising the amino acid sequence described in SEQ ID NO: 7; and

(ii) a peptide comprising a heavy chain or a heavy chain variable region of the antibody of the present invention, the peptide comprising the amino acid sequences described in SEQ ID NOs: 4 to 6, for example, a peptide comprising the amino acid sequence described in SEQ ID NO: 8.

Examples of a peptide comprising anyone of a light chain, a heavy chain and variable regions thereof, of an antibody, the peptide comprising CDR of the antibody 1640, include the following peptides (i) and (ii):

(i) a peptide comprising a light chain or a light chain variable region of the antibody of the present invention, the peptide comprising the amino acid sequences described in SEQ ID NOs: 9 to 11, for example, a peptide comprising the amino acid sequence described in SEQ ID NO: 15; and

(ii) a peptide comprising a heavy chain or a heavy chain variable region of the antibody of the present invention, the peptide comprising the amino acid sequences described in SEQ ID NOs: 12 to 14, for example, a peptide comprising the amino acid sequence described in SEQ ID NO: 16.

A functional antibody can be prepared by linking such peptides with, for example, a linker.

Once a specific anti-serotype E LPS antibody (1656 or 1640) is obtained, those skilled in the art can identify an epitope recognized by the antibody, and prepare various antibodies which bind to the epitope. The present invention also provides an antibody which recognizes an epitope identical to that recognized by any one of the antibody “1656” and the antibody “1640.” It is conceivable that such an antibody has the above-described characteristics of the one of the antibody “1656” and the antibody “1640” (the serotype specificity of binding activity to P. aeruginosa, the opsonic activity, the agglutination activity, and the antibacterial activities against a systemic infection and a pulmonary infection).

The binding of an antibody to P. aeruginosa can be evaluated, for example, by the Whole cell ELISA method, as described in the examples of the present application. Thereby, the range of serotypes of P. aeruginosa strains to which the antibody exhibits a binding activity can be determined. The opsonic activity can be evaluated, for example, by the detection method using, as an index, a fluorescence intensity of human polymorphonuclear leukocytes incorporating FITC-labeled P. aeruginosa, as described in the examples of the present application. Meanwhile, the agglutination activity can be evaluated, for example, as an agglutination titer per amount of IgG, by detecting an agglutinating ability of an antibody against serially diluted bacterial cells, as described in the examples of the present application. Meanwhile, the antibacterial activities against a systemic infection and a pulmonary infection can be evaluated, for example, from a survival rate of model mice to which an antibody is administered, as described in the examples of the present application.

The antibody of the present invention is typically a human antibody. However, by using information on the epitopes identified in the present invention or by using CDR regions or variable regions of the human antibodies identified in the present invention, those skilled in the art can prepare various antibodies such as, for example, chimeric antibodies, humanized antibody and mouse antibodies, in addition to human antibodies, and also can prepare functional fragments of these antibodies. For administration to humans as a medicine, the antibody of the present invention is most preferably a human antibody, from the viewpoint of side effect reduction.

In the present invention, a “human antibody” refers to an antibody of which all regions are originated from human. For the preparation of a human antibody, the methods described in the present examples can be employed. As other methods, for example, a method can be used in which a transgenic animal (for example, a mouse) capable of producing a repertoire of human antibodies by immunization is used. Preparation methods of such human antibodies have been known (for example, Nature, 362: 255-258 (1992), Intern. Rev. Immunol, 13: 65-93 (1995), J. Mol. Biol, 222: 581-597 (1991), Nature Genetics, 15: 146-156 (1997), Proc. Natl. Acad. Sci. USA, 97: 722-727 (2000), Japanese Unexamined Patent Application Publication No. Hei 10-146194, Japanese Unexamined Patent Application Publication No. Hei 10-155492, Japanese Patent No. 2938569, Japanese Unexamined Patent Application Publication No. Hei 11-206387, and International Application Japanese-Phase Publication No. Hei 8-509612, and International Application Japanese-Phase Publication No. Hei 11-505107).

In the present invention, a “chimeric antibody” refers to an antibody obtained by linking a variable region of an antibody of one species with a constant region of an antibody of another species. For example, such a chimeric antibody can be obtained as follows. A mouse is immunized with an antigen. A portion coding an antibody variable part (variable region) which binds to the antigen is cut out from a gene coding a monoclonal antibody of the mouse. The portion is linked with a gene coding a human bone marrow-derived antibody constant part (constant region). These linked genes are incorporated in an expression vector. The expression vector is then introduced into a host which produces a chimeric antibody (Refer to, for example, Japanese Unexamined Patent Application Publication No. Hei 8-280387, U.S. Pat. No. 4,816,397, U.S. Pat. No. 4,816,567, and U.S. Pat. No. 5,807,715). Meanwhile, in the present invention, a “humanized antibody” refers to an antibody obtained by grafting a genome sequence of an antigen-binding site (CDR) of a non-human-derived antibody onto a gene of a human antibody (CDR grafting). Preparation methods of such chimeric antibodies have been known (refer to, for example, EP239400, EP125023, WO90/07861, and WO96/02576). In the present invention, a “functional fragment” of an antibody means a part (a partial fragment) of an antibody, which retains a capability of specifically recognizing an antigen of the antibody from which the part is originated. Specific examples of the functional fragment include Fab, Fab′, F (ab′)2, a variable region fragment (Fv), a disulfide-linked Fv, a single-chain Fv (scFv), sc (Fv) 2, a diabody, a polyspecific antibody, and polymers thereof.

Here, the “Fab” means a monovalent antigen-binding fragment, of a immunoglobulin, formed of a part of one light chain and a part of one heavy chain. The Fab can be obtained by papain-digestion of an antibody, or a recombinant method. The “Fab′” differs from the Fab in that, in Fab′, a small number of residues including one or more cysteines from a hinge region of an antibody are added to the carboxy terminus of a heavy chain CH1 domain. The “F(ab′)2” means a divalent antigen-binding fragment, of an immunoglobulin, made of parts of both light chains and parts of both heavy chains.

The “variable region fragment (Fv)” is a smallest antibody fragment which has a complete antigen recognition and binding site. The Fv is a dimer in which a heavy chain variable region and a light chain variable region are strongly linked by non-covalent bonding. The “single-chain Fv (scFv)” includes a heavy chain variable region and a light chain variable region of an antibody, and in the “single-chain Fv (scFv),” these regions exist in a single polypeptide chain. The “sc(Fv)2” is a single chain obtained by bonding two heavy chain variable regions and two light chain variable regions with a linker or the like. The “diabody” is a small antibody fragment having two antigen binding sites. The fragment include a heavy chain variable region bonded to a light chain variable region in a single polypeptide chain, and each of the regions forms a pair with a complementary region in another chain. The “polyspecific antibody” is a monoclonal antibody which has binding specificity to at least two different antigens. For example, such a polyspecific antibody can be prepared by coexpression of two immunoglobulin heavy chain/light chain pairs, in which two heavy chains have mutually different specificities.

The antibody of the present invention includes antibodies whose amino acid sequences are modified without impairing desirable activities (the binding activity to P. aeruginosa and the broadness thereof or the specificity thereof, the opsonic activity, the agglutination activity, the antibacterial activity against a systemic infection or a pulmonary infection, and/or other biological characteristics). An amino acid sequence variant of the antibody of the present invention can be prepared by introduction of mutation into a DNA coding an antibody chain of the present invention or by peptide synthesis. Such modification includes, for example, substitution, deletion, addition and/or insertion of one or multiple residues in an amino acid sequence of the antibody of the present invention. The modification region of the amino acid sequence of the antibody may be a constant region of a heavy chain or a light chain of the antibody or a variable region (a framework region or CDR) thereof, as long as the resulting antibody has activities which are equivalent to those of an unmodified antibody. It is conceivable that modification on amino acids other than those in CDR has a relatively small effect on binding affinity for an antigen. As of now, there are screening methods of antibodies whose affinity for an antigen is enhanced by modification of amino acids in CDR (PNAS, 102: 8466-8471 (2005), Protein Engineering, Design & Selection, 21: 485-493 (2008), International Publication No. WO2002/051870, J. Biol. Chem., 280: 24880-24887 (2005), and Protein Engineering, Design & Selection, 21: 345-351 (2008)).

The number of amino acids modified are preferably 10 amino acids or less, more preferably 5 amino acids or less, and most preferably 3 amino acids or less (for example, 2 amino acids or less, or 1 amino acid). The modification of amino acids is preferably conservative substitution. In the present invention, the term “conservative substitution” means substitution with a different amino acid residue having a chemically similar side chain. Groups of amino acids having chemically similar amino acid side chains are well known in the technical field to which the present invention pertains. For example, amino acids can be grouped into acidic amino acids (aspartic acid and glutamic acid), basic amino acids (lysine, arginine, and histidine), and neutral amino acids. The neutral amino acids can be sub-classified into amino acids having a hydrocarbon group (glycine, alanine, valine, leucine, isoleucine and proline), amino acids having a hydroxy group (serine and threonine), sulfur-containing amino acids (cysteine and methionine), amino acids having an amide group (asparagine and glutamine), an amino acid having an imino group (proline); and amino acids having an aromatic group (phenylalanine, tyrosine and tryptophan).

The modification on the antibody of the present invention may be modification on post-translational process of the antibody, for example, the change in number of sites of glycosylation or in location of the glycosylation. This can improve, for example, an ADCC activity of the antibody. Glycosylation of an antibody is typically N-linked or O-linked glycosylation. The glycosylation of an antibody greatly depends on a host cell used for expression of the antibody. Alteration in glycosylation pattern can be performed by a known method such as introduction or deletion of a certain enzyme which is related to carbohydrate production (Japanese Unexamined Patent Application Publication No. 2008-113663, U.S. Pat. No. 5,047,335, U.S. Pat. No. 5,510,261, U.S. Pat. No. 5,278,299, International Publication No. WO99/54342). In the present invention, for the purpose of increasing the stability of an antibody or other purposes, an amino acid subjected to deamidation or an amino acid which is adjacent to an amino acid subjected to deamidation may be substituted with a different amino acid to prevent the deamidation. Moreover, a glutamic acid can be substituted with a different amino acid to thereby increase the stability of an antibody. The present invention also provides an antibody thus stabilized.

The polyclonal antibody of the antibodies of the present invention can be obtained as follows. Specifically, an immune animal is immunized with an antigen (LPS, a molecule having a partial structure of LPS, P. aeruginosa on which surface any one of LPS and a molecule having a partial structure of LPS is exposed, or the like). A polyclonal antibody can be obtained by purification of an antiserum obtained from the animal by a conventional method (for example, salting-out, centrifugation, dialysis, column chromatography, or the like). Meanwhile, the monoclonal antibody can be prepared by a standard hybridoma method or a standard recombinant DNA method, in addition to the methods described in the present examples.

A typical example of the hybridoma method is a Kohler & Milstein method (Kohler & Milstein, Nature, 256: 495 (1975)). Antibody-producing cells used in cell fusion process of this method are spleen cells, lymph node cells, peripheral blood leukocytes, and the like of an animal (for example, mouse, rat, hamster, rabbit, monkey or goat) which is immunized with an antigen (LPS, a molecule having a partial structure of LPS, P. aeruginosa on which surface any of LPS and a molecule having a partial structure of LPS is exposed, or the like). Antibody-producing cells obtained by causing an antigen to act, in a culture medium, on any of cells of the above described types and lymphocytes which are isolated from a non-immunized animal in advance can be used. As the myeloma cells, various known cell strains can be used. The antibody-producing cells and the myeloma cells may be originated from different animal species, as long as the antibody-producing cells and the myeloma cells can be fused. However, the antibody-producing cells and the myeloma cells are preferably originated from the same animal species. Hybridomas can be produced by, for example, by cell fusion between spleen cells obtained from a mouse immunized with an antigen and mouse myeloma cells. Thereafter, by screening the hybridomas, a hybridoma which produces a LPS antigen-specific monoclonal antibody can be obtained. The monoclonal antibody against a LPS antigen can be obtained by culturing the hybridoma, or from the ascites in a mammal to which the hybridoma is administered.

The recombinant DNA method is a method with which the above-described antibody of the present invention is produced as a recombinant antibody as follows. A DNA coding the antibody or the peptide of the present invention is cloned from a hybridoma, B cells, or the like. The cloned DNA is incorporated in an appropriate vector, and the vector is introduced into host cells (for example, a mammalian cell strain, Escherichia coli, yeast cells, insect cells, plant cells, or the like) (for example, P. J. Delves, Antibody Production: Essential Techniques, 1997 WILEY, P. Shepherd and C. Dean Monoclonal Antibodies, 2000 OXFORD UNIVERSITY PRESS, Vandamme A. M. et al., Eur. J. Biochem. 192: 767-775 (1990)). For the expression of a DNA cording the antibody of the present invention, DNAs coding a heavy chain and a light chain may be incorporated in expression vectors, respectively, and host cells may be transformed. Alternatively, DNAs coding a heavy chain and a light chain may be incorporated in a single expression vector, and host cells may be transformed (refer to WO94/11523). The antibody of the present invention can be obtained in a substantially pure and homogeneous form by culturing of the above-described host cells, and separation and purification from the host cells or a culture medium. For the separation and purification of the antibody, any method used for standard purification of polypeptide can be used. When a transgenic animal (cattle, goat, sheep, pig or the like) in which an antibody gene is incorporated is produced by a transgenic animal production technique, a large amount of a monoclonal antibody derived from the antibody gene can also be obtained from milk of the transgenic animal.

The present invention also provides a DNA coding the above-described antibody or peptide of the present invention, a vector containing the DNA, host cells having the DNA, and a method of producing an antibody, the method including culturing the host cell and collecting an antibody.

Since the antibody of the present invention has the above-described activities, the antibody of the present invention can be used for prevention or treatment of Diseases associated with P. aeruginosa. Accordingly, the present invention also provides a pharmaceutical composition for use in prevention or treatment of a disease associated with P. aeruginosa, the pharmaceutical composition comprising the antibody of the present invention as an active ingredient, and a method for preventing or treating a disease associated with P. aeruginosa, comprising a step of administering a therapeutically or preventively effective amount of the antibody of the present invention to a mammal including a human. The treatment or prevention method of the present invention can be used for various mammals, in addition to humans, including, for example, dogs, cats, cattle, horses, sheep, pigs, goats, and rabbits.

Examples of the disease associated with P. aeruginosa include systemic infectious diseases, caused by a P. aeruginosa infection including a multidrug resistant P. aeruginosa infection, for example, septicemia, meningitis, and endocarditis. Other examples thereof include: otitis media and sinusitis in the otolaryngologic field; pneumonia, chronic respiratory tract infection, and catheter infection in the pulmonary field; postoperative peritonitis and postoperative infection in a biliary tract or the like in the surgical field; abscess of eyelid, dacryocystitis, conjunctivitis, corneal ulcer, corneal abscess, panophthalmitis, and orbital infection in the ophthalmological field; and urinary tract infections including complicated urinary tract infection, catheter infection, and abscess around the anus in the urologic field. Besides, the examples include burns (including a serious burn and a burn of the respiratory tract), decubital infection, and cystic fibrosis.

A pharmaceutical composition or an agent of the present invention may be used in the form of a composition which uses the antibody of the present invention as an active ingredient, and preferably which contains a purified antibody composition and another component, for example, saline, an aqueous glucose solution or a phosphate buffer.

The pharmaceutical composition of the present invention may be formulated into a preparation in a liquid or lyophilized form as necessary, and may optionally comprise a pharmaceutically acceptable carrier, for example, a stabilizer, a preservative, and an isotonic agent. Examples of the pharmaceutically acceptable carrier includes: mannitol, lactose, saccharose, and human albumin for a lyophilized preparation; and saline, water for injection, a phosphate buffer, and aluminum hydroxide for a liquid preparation. However, the examples are not limited thereto.

An administration may differ depending on the age, weight, gender, and general health state of an administration target. The administration can be carried out by any administration route of oral administration and parenteral administration (for example, intravenous administration, intraarterial administration, and local administration). However, parenteral administration is preferable.

The dose of the pharmaceutical composition varies depending on the age, weight, sex, and general health state of a patient, the severity of a P. aeruginosa infection and components of an antibody composition to be administered. The dose of the antibody composition of the present invention is generally 0.1 to 1000 mg, and preferably 1 to 100 mg, per kg body weight per day for an adult in a case of intravenous administration.

The pharmaceutical composition of the present invention is preferably administered in advance to a patient who may develop a P. aeruginosa infection.

Since the antibody of the present invention binds to LPS exposed on the cell surface of P. aeruginosa, the antibody of the present invention can also be used as a P. aeruginosa infection diagnostic agent.

When the antibody of the present invention is prepared as a diagnostic agent, the diagnostic agent can be obtained in any dosage form by adopting any means suitable for the purpose. For example, ascites, a culture medium containing an antibody of interest, or a purified antibody is measured for the antibody titer and appropriately diluted with PBS (phosphate buffer containing saline) or the like; thereafter, a preservative such as 0.1% sodium azide is added thereto. Alternatively, the antibody of the present invention adsorbed to latex or the like is determined for the antibody titer and appropriately diluted, and a preservative is added thereto for use. The antibody of the present invention bound to latex particles as described above is one of preferable dosage forms as a diagnostic agent. As the latex in this case, appropriate resin materials, for example, latex of polystyrene, polyvinyl toluene, or polybutadiene, are suitable.

According to the present invention, provided is a diagnosis method for a P. aeruginosa infection using the antibody of the present invention. The diagnosis method of the present invention can be carried out by collecting a biological sample such as expectoration, a lung lavage fluid, pus, a tear, blood, or urine from mammals, including a human, which may have developed a P. aeruginosa infection, subsequently bringing the collected sample into contact with the antibody of the present invention, and determining whether or not an antigen-antibody reaction occurs.

According to the present invention, provided is a kit for detecting the presence of P. aeruginosa, the kit comprising at least the antibody of the present invention.

The antibody of the present invention may be labeled. This kit for detection detects the presence of P. aeruginosa by detecting the antigen-antibody reaction.

Thus, the detection kit of the present invention can further include various reagents for carrying out the antigen-antibody reaction, for example, a secondary antibody, a chromogenic reagent, a buffer, instructions, and/or an instrument used in an ELISA method, and the like, if desired.

### EXAMPLES

Hereinafter, the present invention will be described more specifically on the basis of examples. However, the present invention is not limited to these examples.

### Example 1

### Cloning of Anti-LPS Antibody

### (1) Blood Donor Recruitment

250 ml blood samples were collected from Cystic Fibrosis Patients having a chronic PA lung infection and from healthy volunteers. Donors were generally of good health and represented a wide range in age, years of chronic PA infection, as well as immune response status. Additional inclusion criteria were an age above 18 years, a body weight above 50 kilograms and normal hemoglobin levels. All donations were approved by the Danish National Committee on Biomedical Research Ethics.

The following types of analyses were performed on each blood samples: i) FACS analyses to determine the amount of circulating plasma blasts and plasma cells, ii) ELISPOT analyses to determine the amount of circulating antibody producing cells specific for particular LPS antigens, iii) ELISA analyses to determine the presence of specific immunoglobulin towards particular LPS antigens.

Donor samples with a high percentage of plasma blasts specific for LPS antigens were chosen for the Symplex procedure (refer to WO2005/042774) described below.

### (2) FACS Sorting of Human Plasmablasts

The starting materials for this procedure were MACS-purified CD19 positive B-cells. These cells were normally stored frozen and then a fraction was thawed before each sorting. Viable plasma blasts were identified by staining cells for CD19, CD38, the lambda-light chain and dead cells.

Freshly thawed cells were washed twice with 4 ml FACS PBS, diluted to 1×10⁶ cells per 40 μl FACS PBS. Per 1×10⁶ cells the following reagents was added: 10 μl CD19-FITC, 20 μl CD38 APC and 10 μl Lambda-PE at 4° C. and left for 20 minutes in the dark on ice. Samples were washed twice with 2 ml FACS buffer and resuspended in 1 ml FACS PBS whereafter propidium iodide was added (1:100). The cell-suspension was filtered through a 50 μm Syringe falcon (FACS filter), and was ready for sorting directly into Symplex PCR plates (see next section). After sorting, PCR plates were centrifuged at 300×g for 1 minutes and stored at −80° C. for later use.

### (3) Linkage of Cognate VH and VL Pairs

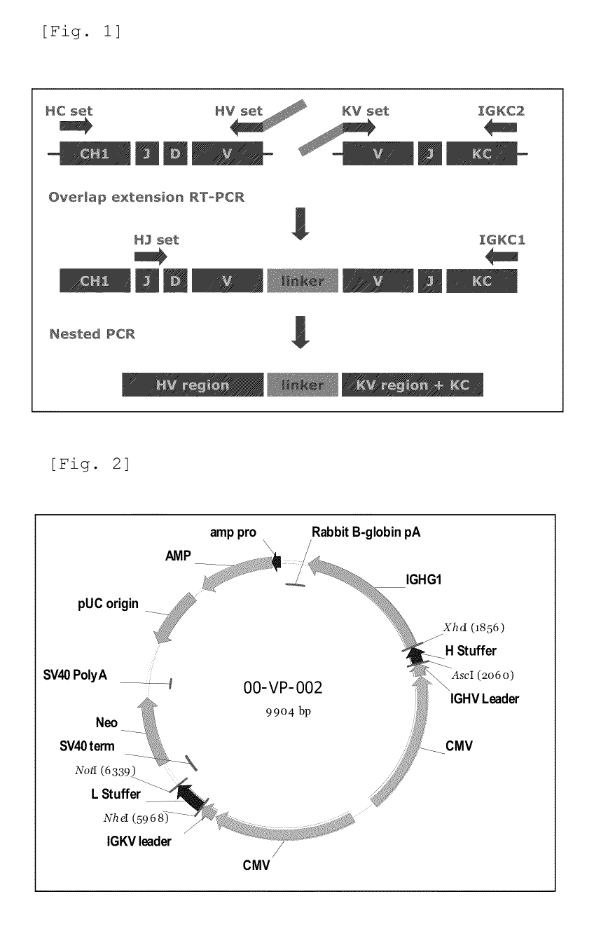

In order to pair sequences coding a heavy chain variable region (VH) and a light chain variable region (VL) which were originated form the same B cell, the sequences coding the VH and the VL were linked on a single cell gated as plasma cells. The procedure utilized a two step PCR procedure based on a one-step multiplex overlap-extension RT-PCR followed by a nested PCR. The primer mixes used in the present example only amplify Kappa light chains. The principle for linkage of cognate VH and VL sequences was showed in FIG. 1.

The 96-well PCR plates produced were thawed and the sorted cells served as template for the multiplex overlap-extension RT-PCR. The sorting buffer added to each well before the single-cell sorting contained reaction buffer (OneStep RT-PCR Buffer; Qiagen), primers for RT-PCR (refer to Table 2) and RNase inhibitor (RNasin, Promega). This was supplemented with OneStep RT-PCR Enzyme Mix (25× dilution; Qiagen) and dNTP mix (200 μM each) to obtain the given final concentration in a 20-μl reaction volume.

TABLE 2

| | | |
|---|---|---|
| | | Final |
| Symplex ™ | | concentration |
| primer mix | Sequence (5′-3′) | (pmol/μL) |

Multiplex PCR

| | | |
|---|---|---|
| KC | | |
| IGKC2 | ATATATATGCGGCCGCTTATTAACACTCTCCCCTGTTG (SEQ ID NO: 31) | 51.25 |
| HC set | | |
| IGHG | GACSGATGGGCCCTTGGTGG (SEQ ID NO: 32) | 51.25 |
| IGHA | GAGTGGCTCCTGGGGGAAGA (SEQ ID NO: 33) | 51.25 |
| HV set | | |
| HV1 | TATTCCCATGGCGCGCCCAGRTGCAGCTGGTGCART (SEQ ID NO: 34) | 10.24 |
| HV2 | TATTCCCATGGCGCGCCSAGGTCCAGCTGGTRCAGT (SEQ ID NO: 35) | 10.24 |
| HV3 | TATTCCCATGGCGCGCCCAGRTCACCTTGAAGGAGT (SEQ ID NO: 36) | 10.24 |
| HV4 | TATTCCCATGGCGCGCCSAGGTGCAGCTGGTGGAG (SEQ ID NO: 37) | 10.24 |
| HV5 | TATTCCCATGGCGCGCCCAGGTGCAGCTACAGCAGT (SEQ ID NO: 38) | 10.24 |
| HV6 | TATTCCCATGGCGCGCCCAGSTGCAGCTGCAGGAGT (SEQ ID NO: 39) | 10.24 |
| HV7 | TATTCCCATGGCGCGCCGARGTGCAGCTGGTGCAGT (SEQ ID NO: 40) | 10.24 |
| HV8 | TATTCCCATGGCGCGCCCAGGTACAGCTGCAGCAGTC (SEQ ID NO: 41) | 10.24 |
| KV set | | |
| KV1 | GGCGCGCCATGGGAATAGCTAGCCGACATCCAGWTGACCCAGTCT (SEQ ID NO: 42) | 10.24 |
| KV2 | GGCGCGCCATGGGAATAGCTAGCCGATGTTGTGATGACTCAGTCT (SEQ ID NO: 43) | 10.24 |
| KV3 | GGCGCGCCATGGGAATAGCTAGCCGAAATTGTGWTGACRCAGTCT (SEQ ID NO: 44) | 10.24 |
| KV4 | GGCGCGCCATGGGAATAGCTAGCCGATATTGTGATGACCCACACT (SEQ ID NO: 45) | 10.24 |
| KV5 | GGCGCGCCATGGGAATAGCTAGCCGAAACGACACTCACGCAGT (SEQ ID NO: 46) | 10.24 |
| KV6 | GGCGCGCCATGGGAATAGCTAGCCGAAATTGTGCTGACTCAGTCT (SEQ ID NO: 47) | 10.24 |

Nested PCR

| | | |
|---|---|---|
| KC | | |
| IGKC1 | ACCGCCTCCACCGGCGGCCGCTTATTAACACTCTCCCCTGTTGAAGCTCTT (SEQ ID NO: 48) | 51.25 |
| HJ set | | |
| IGHJ 1-2 | GGAGGCGCTCGAGACGGTGACCAGGGTGCC (SEQ ID NO: 49) | 51.25 |
| IGHJ 3 | GGAGGCGCTCGAGACGGTGACCATTGTCCC (SEQ ID NO: 50) | 51.25 |
| IGHJ 4-5 | GGAGGCGCTCGAGACGGTGACCAGGGTTCC (SEQ ID NO: 51) | 51.25 |
| IGHJ 6 | GGAGGCGCTCGAGACGGTGACCGTGGTCCC (SEQ ID NO: 52) | 51.25 |

The plates were incubated for 30 minutes at 55° C. to allow for reverse transcription of the RNA from each cell. After the reverse transcription, the plates were subjected to the following PCR cycle: 10 minutes at 94° C., 35×(40 seconds at 94° C., 40 seconds at 60° C., 5 minutes at 72° C.), 10 minutes at 72° C.

The PCR reactions were performed in H20BIT Thermal cycler (ABgene) with a Peel Seal Basket for 24 96-well plates to facilitate a high-throughput. The PCR plates were stored at −20° C. after cycling.

For the nested PCR step, 96-well PCR plates were prepared with the following mixture in each well (20-μl reactions) to obtain the given final concentration: 1× FastStart buffer (Roche), dNTP mix (200 μM each), nested primer mix (see Table 1), Phusion DNA Polymerase (0.08 U; Finnzymes) and FastStart High Fidelity Enzyme Blend (0.8 U; Roche). As template for the nested PCR, 1 μl was transferred from the multiplex overlap-extension PCR reactions. The nested PCR plates were subjected to the following thermo cycling: 35×(30 seconds at 95° C., 30 seconds at 60° C., 90 seconds at 72° C.), 10 minutes at 72° C.

Randomly selected reaction products were analyzed on a 1% agarose gel to verify the presence of an overlap-extension fragment of approximately 1050 base pairs (bp). The plates were stored at −20° C. until further processing of the PCR fragments. The repertoires of linked VH and VL coding pairs from the nested PCR were pooled, without mixing pairs from different donors, and were purified by preparative 1% agarose gel electrophoresis.

(4) Insertion of Cognate VH and VL Coding Sequence Pairs into a Screening Vector

In order to identify antibodies with binding specificity to LPS, the VH and VL coding sequences obtained were expressed as full-length antibodies. This involved insertion of the repertoire of VH and VL coding pairs into an expression vector and transfection into a host cell.

A two-step cloning procedure was employed for generation of a repertoire of expression vectors containing the linked VH and VL coding pairs. Statistically, if the repertoire of expression vectors contains ten times as many recombinant plasmids as the number of cognate paired VH and VL PCR products used for generation of the screening repertoire, there is 99% likelihood that all unique gene pairs are represented. Thus, if 400 overlap-extension V-gene fragments were obtained, a repertoire of at least 4000 clones was generated for screening.

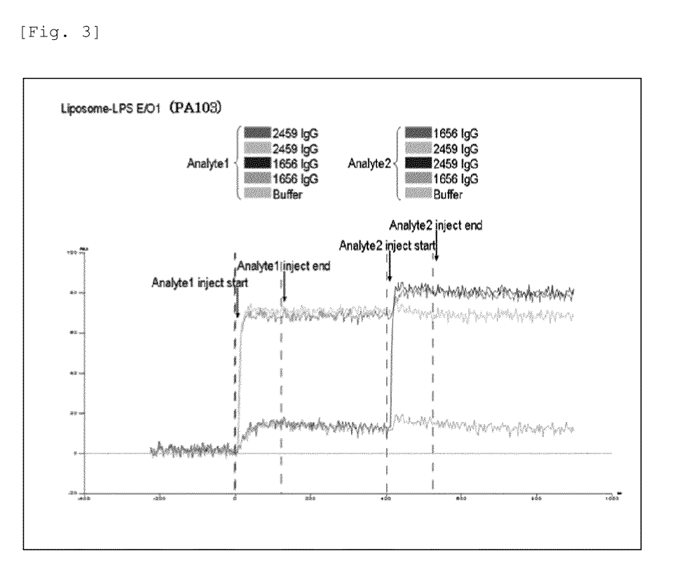

Briefly, the purified PCR product of the repertoires of linked VH and VL coding pairs were cleaved with XhoI and NotI DNA endonucleases at the recognition sites introduced into the termini of PCR products. The cleaved and purified fragments were ligated into an XhoI/NotI digested mammalian IgG expression vector, OO-VP-002 (FIG. 2) by standard ligation procedures. The ligation mix was electroporated into E. coli and added to 2xYT plates containing the appropriate antibiotic and incubated at 37° C. over night. The amplified repertoire of vectors was purified from cells recovered from the plates using standard DNA purification methods (Qiagen).

The plasmids were prepared for insertion of promoter-leader fragments by cleavage using AscI and NheI endonucleases. The restriction sites for these enzymes were located between the VH and VL coding gene pairs. Following purification of the vector, an AscI-NheI digested bi-directional mammalian promoter-leader fragment was inserted into the AscI and NheI restriction sites by standard ligation procedures. The ligated vector was amplified in E. coli and the plasmid was purified using standard methods. The generated repertoire of screening vectors was transformed into E. coli by conventional procedures. Colonies obtained were consolidated into 384-well master plates and stored. The number of colonies transferred to the 384-well plates exceeded the number of used PCR products by at least 3-fold, thus giving 95% likelihood for presence of all unique V-gene pairs obtained.

M166 was expressed as a chimeric IgG antibody. The variable gene amino acid sequences of M166 originate from a murine antibody specific for the Pseudomonas aeruginosa PcrV protein as described in the patent WO2002/064161. Variable genes were synthesized at GENEART AG (BioPark, Josef-Engert-Str. 11, 93053 Regensburg, Germany) and in that process linking the murine light chain variable gene to the human kappa constant gene. The murine heavy chain variable gene and the chimeric light chain gene were inserted into an expression vector harboring the remaining part of the human heavy chain constant genes as well as elements required for gene expression in mammalian cells.

### (5) Expression of Symplex Repertoires

The bacteria colonies on the master plates were planted in a culture medium in 384-well plates, and cultured overnight. A DNA for transfection was prepared from each well using TempliPhi DNA amplification Kit (Amersham Biosciences) in accordance of the manual thereof. On the day before the transfection, Flp-In™-CHO cells (Invitrogen) were planted in the 384-well plates at 3000 cells per well (in 20 μl of culture medium). The amplified DNAs were introduced into cells using FuGENE 6 (Roche) in accordance with the manual thereof. After 3-day culture, the supernatant containing full-length antibodies was collected, and stored for antigen specificity screening.

### (6) Screening for Binding to LPS

By an ELISA method, screening of antibody library was performed using the binding to a mixture of purified LPS molecules isolated from related P. aeruginosa type strains as an index. A Nunc MaxiSorp 384-well plate was coated at 4° C. overnight with a LPS mixture (containing 6 serotypes per assay at maximum) obtained by diluting a mixture of purified LPS molecules with a 50 mM carbonate buffer (pH: 9.6) so that 10 μg/ml of purified LPS of each LPS serotype was contained. The well plate was blocked by 50 μl of PBS-T (PBS+0.05% Tween) containing 2% of skimmed milk (SM), and then washed once with PBS-T. 15 μl of an antibody supernatant was added into each well and incubation at room temperature for 1.5 hours was performed. Then, the plate was washed once with PBS-T. To detect antibodies binding to the wells, a secondary antibody (HRP-Goat-anti-human IgG, Jackson) diluted 10.000-fold with 2% SM-PBS-T was added to each well, then incubation was performed at room temperature for 1 hour. The plate was washed once with PBS-T, and then 25 μl of a substrate (Kemen-tec Diagnostics, catalog No. 4390) was added to each well. Then, incubation was performed for 5 minutes. After the incubation, 25 μl of 1 M sulfuric acid was added to terminate the reaction. A specific signal was detected by 450 nm-ELISA reader.

### (7) Sequence Analysis and Clone Selection

The clones identified as LPS-specific in ELISA were retrieved from the original master plates (384-well format) and consolidated into new plates. DNA was isolated from the clones and submitted for DNA sequencing of the V-genes. The sequences were aligned and all the unique clones were selected. Multiple alignments of obtained sequences revealed the uniqueness of each particular clone and allowed for identification of unique antibodies. Multiple genetically distinct antibody sequence clusters were identified. Each cluster of related sequences have probably been derived through somatic hypermutations of a common precursor clone. Overall, one to two clones from each cluster was chosen for validation of sequence and specificity.

### (8) Sequence and Specificity Validation

In order to validate the antibody encoding clones, DNA plasmid was prepared and transfection of FreeStyle CHO—S cells (Invitrogen) in 2-ml scale was performed for expression. The supernatant were harvested 96 hours after transfection. Expression levels were estimated with standard anti-IgG ELISA, and the specificity was determined by LPS-specific ELISA.

### (9) Identified Antibody

As a result of the above, identified anti-LPS antibodies and the sequences of CDRs and variable regions of the identified anti-LPS antibodies are as follows. Note that the sequences of constant regions of the identified anti-LPS antibodies are as described in WO 2005/042774.

### <Anti-Serotype E LPS Antibody>

“1656”
SEQ ID NOs: 1 to 3 . . . amino acid sequences of light chain CDRs 1 to 3
SEQ ID NOs: 4 to 6 . . . amino acid sequences of heavy chain CDRs 1 to 3
SEQ ID NO: 7 . . . an amino acid sequence of a light chain variable region
SEQ ID NO: 8 . . . an amino acid sequence of a heavy chain variable region
SEQ ID NO: 25 . . . a base sequence of a light chain variable region
SEQ ID NO: 26 . . . a base sequence of a heavy chain variable region
“1640”
SEQ ID NOs: 9 to 11 . . . amino acid sequences of light chain CDRs 1 to 3
SEQ ID NOs: 12 to 14 . . . amino acid sequences of heavy chain CDRs 1 to 3
SEQ ID NO: 15 . . . an amino acid sequence of a light chain variable region
SEQ ID NO: 16 . . . an amino acid sequence of a heavy chain variable region
SEQ ID NO: 27 . . . a base sequence of a light chain variable region
SEQ ID NO: 28 . . . a base sequence of a heavy chain variable region

### <Broadly Reactive Anti-LPS Antibody>

“2459”
SEQ ID NOs: 17 to 19 . . . amino acid sequences of light chain CDRs 1 to 3
SEQ ID NOs: 20 to 22 . . . amino acid sequences of heavy chain CDRs 1 to 3
SEQ ID NO: 23 . . . an amino acid sequence of a light chain variable region
SEQ ID NO: 24 . . . an amino acid sequence of a heavy chain variable region
SEQ ID NO: 29 . . . a base sequence of a light chain variable region
SEQ ID NO: 30 . . . a base sequence of a heavy chain variable region

### Example 2

### Analysis of Anti-Serotype E LPS Antibody

### (1) Purification of LPS

Each P. aeruginosa strain of various serotypes shown in Table 3 was suspended in 5 ml of a LB medium. Using this bacterial cell suspension, 1- to 10⁴-fold diluted liquids were prepared by 10-fold serial dilution. These diluted liquids were shaken at 37° C. for 6 hours, for culturing. After the culturing, a bacterial liquid was taken from a diluted liquid which had the largest dilution factor among diluted liquids in which bacterial growth was observed. This bacterial liquid was suspended in a separately prepared LB medium with a dilution factor of 1000, and then shaken at 37° C. overnight for culturing. After the culturing, the liquid was subjected to centrifugation at 5000×g for 20 minutes, and thereby bacterial cells were collected. The weight of the bacterial cells was measured, and then purified water was added to the bacterial cells at 120 mg/ml, in terms of wet weight. Moreover, an equal amount of a 90% solution of phenol (NACALAI TESQUE, INC.) warmed to 68° C. beforehand was added to the bacterial cells, and the mixture was stirred for 20 minutes. Thereafter, the mixture was heated in a water bath at 68° C. for 20 minutes with occasional stirring. Then, after cooling, the mixture was subjected to centrifugation at 5000×g for 20 minutes. The aqueous layer was collected, dialyzed against purified water, and lyophilized. The resulting product was used as each LPS.

### (2) A-band LPS Purification

LPS G extracted in the above (1) from a P. aeruginosa strain ATCC 27584 of serotype G was used as a raw material. This LPS was again suspended in water for injection, and ultracentrifugation (40000 rpm, 3 hr) was repeated twice to remove nucleic acid. The collected precipitates were lyophilized. The LPS G obtained here was passed through a gel filtration column (HiPrep 26/60 Sephacryl S-200 HR, GE healthcare bioscience, 17-1195-01) for coarse fractionation. For the purification operation, AKTA explore 10S (GE healthcare bioscience) was used. As the mobile phase, a 20 mM Tris-HCl buffer (NACALAI TESQUE, INC., 35406-75) (pH: 8.3) containing 0.2% sodium deoxycholate (NACALAI TESQUE, INC., 10712-54), 0.2 M NaCl (NACALAI TESQUE, INC., 31319-45) and 5 mM EDTA (NACALAI TESQUE, INC., 15105-35) was used. For detection, a differential refractometer (SHIMAZU, RID-10A) was used. The obtained roughly purified fraction was dialyzed against purified water overnight, and then lyophilized. The lyophilized material was again suspended in a 0.5 M NaCl solution, and a 10-fold amount of ethanol was added thereto to thereby cause LPS to be precipitated. The precipitates were again washed with 70% ethanol, to remove the remaining surfactant. Thereafter, the LPS was lyophilized, suspended in a solution of 0.1 N NaOH (NACALAI TESQUE, INC., 31511-05) and 0.2 MNaBH₄ (NACALAI TESQUE, INC., 31228-22), and reacted at 37° C. for 24 hr. Thereby, only B-band LPS contained was decomposed according to the method described in Eur. J. BioChem. 167, 203-209 (1987). This reaction liquid was neutralized with a 1% acetic acid (NACALAI TESQUE, INC., 00211-95), concentrated by ultrafiltration (Amicon Ultra-15, MWCO 10000, Millipore), and then subjected again to a gel filtration column (Superdex peptide 10/300 GL, GE healthcare bioscience, 17-5176-01). Fractions eluted using PBS(−) (Sigma-Aldrich Corporation, D1408) as the mobile phase were collected. Thereafter, buffer replacement with purified water and concentration were performed by ultrafiltration. Then, lyophilization was performed to obtain purified A-band LPS.

### (3) Western Blotting and Whole Cell ELISA

### —Western Blotting—

Each of the LPSs obtained from the ATCC strains of various serotypes prepared in Example 2 (1) and the A-band LPS purified in Example 2(2), which were lyophilized, was dissolved in PBS so as to be 1 mg/ml. The solution was mixed with an equal amount of a sample buffer (62.5 mM Tris-HCL (pH: 6.8), 5% 2-mercaptoethanol, 2% SDS, 20% glycerol, 0.005% bromophenol blue), and heated at 100° C. for 10 minutes before use. 10 μl of a LPS was added in each well of 16 well-type 5-20% or 15% SDS-PAGE (XV PANTERA Gel, DRC), and then electrophoresed for 15 minutes. After transfer to a nitrocellulose membrane using a semidry blotting apparatus (AE-6677, ATTO corporation) or a dry gel blotting apparatus (iBlotdry gel blotting system, Invitrogen), blocking was performed at room temperature for 30 minutes using Immunoblock™ (Dainippon Sumitomo Pharma Co., Ltd.). The antibody sample was diluted to 3 μg/ml with 5% Immunoblock™ in TBST (Tris-Buffered Saline containing 0.05% Tween 20), and reacted with the transfer membrane at 4° C. for a day and a night. After washed with TBST for 10 minutes three times, the transfer membrane was immersed in a reaction liquid obtained by diluting a goat anti-human IgG (Fc) antibody HRP conjugate (Kirkegaard & Perry Laboratories, Inc.) with 5% Immunoblock™ in TBST (1:5000), and reaction was performed at 37° C. for 1 hour. Then, after the transfer membrane was washed with TBST for 10 minutes three times, reaction was performed at room temperature for 2 minutes according to the manual of ECL plus Western Blotting Detection System (GE Healthcare, Code: RPN2132). Chemiluminescence was detected by a FLA-3000 fluorescent image analyzer (FUJIFILM Corporation).

Table 3 shows the results. On each membrane to which the antibody 1640 or the antibody 1656 was added as the primary antibody, multiple bands presumably corresponding to B-band LPSs including O antigens were observed only from the low molecular weight region to the high molecular weight region of the LPS obtained from the clinically frequently encountered serotype E strain, out of the LPSs obtained from the ATCC strains of 11 serotypes. When LPS obtained from another serotype E strain ATCC 33358 was used, the antibody 1656 taken as a representative exhibited the same results. Moreover, the antibody 1656 did not show any reactivity to the purified A-band LPS. Accordingly, it was confirmed that these antibodies specifically recognized B-band LPS of serotype E LPSs.

TABLE 3

| ATCC | Serotype | 1640 | 1656 |
|---|---|---|---|
| 27577 | A/O3 | ND | ND |
| 27578 | B/O2 | ND | ND |
| BAA-47 | B/O5 | ND | ND |
| 27317 | C/O8 | ND | ND |
| 27580 | D/O9 | ND | ND |
| 29260 | E/O11 | B band | B band |
| 33358 | E/O11 | NT | B band |
| 27582 | F/O4 | ND | ND |
| 27584 | G/O6 | ND | ND |
| 27316 | H/O10 | ND | ND |
| 27586 | I/O1 | ND | ND |
| 21636 | M | ND | ND |

NT: not tested
ND: not detected

### —Whole Cell ELISA (1)—

Bacterial suspensions used for immobilization were original bacterial suspensions which were prepared by washing, with PBS, bacterial suspensions of P. aeruginosa strains of various serotypes cultured overnight in LB media, and resuspending the washed materials so that the absorbance at 595 nm of each 10-fold diluted bacterial suspensions was 0.20 to 0.23. The bacterial suspensions were placed at 100 μl per well of a 96 well ELISA plate (F96 MaxiSorp Nunc-Immuno Plate, Nalge Nunc International K. K.), and immobilization was performed at 4° C. overnight. Thereafter, washing was performed once with 200 of TBS. A blocking buffer (TBS containing 2% bovine serum albumin) was added to each of the wells, and blocking was performed for 30 minutes at room temperature. Then, 100 of the anti-serotype E LPS antibodies 1640 and 1656 diluted (1 μg/ml) with a sample buffer (TBS containing 1% bovine serum albumin) was added to each of the wells, and reaction was performed at 37° C. for 2 hours. Thereafter, washing was performed three times each time with 200 of a washing buffer (TBS containing 0.05% Tween 20). 100 of a secondary antibody, goat anti-human IgG (Fc) antibody HRP conjugate (Kirkegaard & Perry Laboratories, Inc.), diluted 10000-fold with the sample buffer was added to each of the wells, and reaction was performed at 37° C. for 1 hour. Thereafter, washing was performed three times with the washing buffer. 100 of a chromogenic substrate (TMB Microwell Peroxidase substrate System, Kirkegaard & Perry Laboratories, Inc.) was added to each of the wells, and reaction was performed in a dark place. Then, the enzymatic reaction was stopped with a 1 M solution of phosphoric acid, and the absorbance at 450 nm was measured. Table 4 shows the results. It was confirmed that, when an absorbance greater than 0.25 was judged as positive, the antibody 1640 and the antibody 1656 specifically bound to a serotype E strain.

TABLE 4

| ATCC | Serotype | 1640 | 1656 | Venilon |
|---|---|---|---|---|
| 27577 | A/O3 | 0.005 | 0.005 | 0.011 |
| 27578 | B/O2 | −0.004 | 0.000 | 0.011 |
| BAA-47 | B/O5 | −0.013 | −0.014 | −0.005 |
| 33353 | C/O7 | 0.001 | 0.000 | 0.003 |
| 27580 | D/O9 | 0.003 | 0.003 | 0.006 |
| 29260 | E/O11 | 1.245 | 1.337 | 0.010 |
| 27582 | F/O4 | −0.001 | 0.002 | 0.009 |
| 27584 | G/O6 | −0.002 | −0.004 | 0.007 |
| 27316 | H/O10 | 0.002 | 0.001 | 0.002 |
| 27586 | I/O1 | −0.012 | −0.011 | −0.010 |
| 21636 | M | −0.001 | 0.000 | 0.011 |

### —Whole Cell ELISA (2)—

Whole cell ELISA was performed on the anti-serotype E LPS antibody 1656 (1.0 μg/ml), using 31 strains in total, which additionally included various serotype strains. Table 5 shows the results. The criteria were as follows: a case with an absorbance of less than 0.25 was marked with −, a case with an absorbance which was 0.25 or more but less than 0.5 was marked with +, a case with an absorbance which was 0.5 or more but less than 0.75 was marked with ++, and a case with an absorbance of 0.75 or more was marked with +++. In such a case, a human immunoglobulin preparation, Venilon (TEIJIN PHARMA LIMITED), which was a control, exhibited no binding capability to the 31 strains examined. In contrast, the antibody 1656 had +++ and ++ only for serotype E strains, and had − for all the strains of the other serotypes, exhibiting a specificity to serotype E strains.

TABLE 5

| | | | |
|---|---|---|---|
| ATCC | Serotype | 1656 | Venilon |

| | | | | | |
|---|---|---|---|---|---|
| 27577 | A/O3 | −0.003 | − | 0.025 | — |
| 33350 | A/O3 | 0.007 | − | 0.008 | — |
| 27578 | B/O2 | 0.008 | − | 0.032 | — |
| 33349 | B/O2 | −0.003 | − | 0.068 | — |
| BAA-47 | B/O5 | −0.009 | − | 0.032 | — |
| 33352 | B/O5 | −0.006 | − | 0.045 | — |
| 33363 | B/O16 | 0.002 | − | 0.043 | — |
| 43732 | B/O20 | −0.005 | − | 0.155 | — |
| 33353 | C/O7 | −0.007 | − | 0.003 | — |
| 27317 | C/O8 | 0.003 | − | 0.029 | — |
| 33355 | C/O8 | 0.003 | − | 0.015 | — |
| 27580 | D/O9 | 0.007 | − | 0.020 | — |
| 33356 | D/O9 | 0.005 | − | 0.013 | — |
| 29260 | E/O11 | 0.984 | +++ | 0.028 | — |
| 33358 | E/O11 | 0.710 | ++ | 0.031 | — |
| 27582 | F/O4 | −0.010 | − | 0.007 | — |
| 33351 | F/O4 | 0.006 | − | 0.018 | — |
| 27584 | G/O6 | 0.031 | − | 0.037 | — |
| 33354 | G/O6 | 0.009 | − | 0.026 | — |
| 27316 | H/O10 | −0.010 | − | 0.008 | — |
| 33357 | H/O10 | −0.001 | − | 0.014 | — |
| 27586 | I/O1 | 0.000 | − | 0.012 | — |
| 33348 | I/O1 | 0.008 | − | 0.009 | — |
| 33362 | J/O15 | −0.002 | − | 0.016 | — |
| 33360 | K/O13 | 0.001 | − | 0.012 | — |
| 33361 | K/O14 | 0.011 | − | 0.024 | — |
| 33359 | L/O12 | 0.008 | − | 0.023 | — |
| 21636 | M | 0.003 | − | 0.028 | — |
| 33364 | N/O17 | 0.050 | − | 0.020 | — |
| 43390 | O18 | 0.007 | − | 0.014 | — |
| 43731 | O19 | 0.004 | − | 0.009 | — |

### —Whole Cell ELISA (3)—

The binding capability of the anti-serotype E LPS antibody 1656 of the present invention to nine strains of multi-drug resistant P. aeruginosa (MDRP) of serotype E/O11 possessed by MEIJI SEIKA KAISHA, LTD. was examined. The criteria were as follows: a case with an absorbance of less than 0.25 was marked with −, a case with an absorbance which was 0.25 or more but less than 0.5 was marked with +, a case with an absorbance which was 0.5 or more but less than 0.75 was marked with ++, and a case with an absorbance of 0.75 or more was marked with +++. A human immunoglobulin preparation, Venilon (TEIJIN PHARMA LIMITED, 1.0 μg/ml), which was a control, exhibited no binding capability at all to the nine strains tested. In contrast, the antibody 1656 (1.0 μg/ml) was evaluated as + for two strains, ++ for five strains, and +++ for two strains, exhibiting a strong binding capability also to the MDRP, despite the presence of the antimicrobial resistance. Table 6 shows the results.

TABLE 6

| | | | |
|---|---|---|---|
| Strain | serotype | 1656 | Venilon |

| | | | | | |
|---|---|---|---|---|---|
| MSC06120 | E/O11 | 0.777 | +++ | 0.016 | — |
| MSC17660 | E/O11 | 0.630 | ++ | 0.046 | — |
| MSC17661 | E/O11 | 0.301 | + | 0.099 | — |
| MSC17662 | E/O11 | 0.320 | + | 0.149 | — |
| MSC17667 | E/O11 | 0.662 | ++ | 0.060 | — |
| MSC17671 | E/O11 | 0.801 | +++ | 0.024 | — |
| MSC17693 | E/O11 | 0.632 | ++ | 0.022 | — |
| MSC17727 | E/O11 | 0.579 | ++ | 0.004 | — |
| MSC17728 | E/O11 | 0.643 | ++ | 0.017 | — |

### (4) Cross-Reactivity Test

To test cross-reaction of the anti-serotype E LPS antibody 1656 (1.0 μg/ml), whole cell ELISA was performed using various Gram-negative and Gram-positive pathogenic bacteria in the same method as in the above (1). Table 7 shows the results. The anti-serotype E LPS antibody 1656 specifically recognized and bound strongly to the serotype E/O11 ATCC 29260 strain, but did not react with other bacterial strains.

TABLE 7

| | 1656 | Venilon | Synagis |
|---|---|---|---|
| P. aeruginosa ATCC 27577 (A/O3) | 0.007 | 0.020 | 0.006 |
| P. aeruginosa ATCC BAA-47 (B/O5) | 0.006 | 0.016 | 0.008 |
| P. aeruginosa ATCC 29260 (E/O11) | 0.530 | 0.014 | 0.004 |
| P. aeruginosa ATCC 27584 (G/O6) | 0.004 | 0.013 | 0.002 |
| P. aeruginosa ATCC 27586 (I/O1) | 0.011 | 0.015 | 0.003 |
| P. aeruginosa ATCC 21636 (M) | 0.004 | 0.016 | 0.001 |
| P. alcaligenes ATCC 14909 | 0.024 | 0.021 | 0.006 |
| P. aureofaciens ATCC 13985 | 0.013 | 0.017 | 0.005 |
| P. chlororaphis ATCC 9446 | 0.019 | 0.007 | 0.003 |
| Acinetobacter baumannii ATCC | −0.008 | 0.012 | −0.005 |
| BAA-1710 | | | |
| Stenotrophomonas maltophilia ATCC | 0.020 | 0.021 | −0.002 |
| 13637 | | | |
| Burkholderia cepacia ATCC 25416 | 0.008 | 0.011 | −0.001 |
| Bacillus subtillis ATCC 6633 | 0.010 | 0.047 | −0.002 |
| Escherichia coli ATCC 25922 | 0.015 | 0.025 | 0.001 |
| Klebsiella pneumoniae ATCC 700603 | 0.001 | 0.018 | −0.004 |

### (5) Agglutination Activity

Using a P. aeruginosa ATCC 29260 strain (serotype E/O11), the agglutination activity of the antibody 1656 was measured. This strain was cultured on a trypticase soy agar medium at 37° C. overnight. Then, after several colonies were suspended in a LB medium, the medium was shaken at 37° C. overnight for culturing. The bacterial culture was washed with PBS and resuspended in PBS. Then, a phosphate buffer containing 4% paraformaldehyde (Wako Pure Chemical Industries, Ltd.) was added thereto, and inactivation treatment was performed for 30 minutes or more. This treated product was used for the test. The inactivated ATCC 29260 strain was suspended in PBS so as to be 2 mg/ml of protein concentration. The antibody 1656 (concentration of IgG in the original liquid: 2.69 mg/ml) was serially diluted with PBS. Equal amounts (8 μl) of the inactivated ATCC 29260 strain suspension and the serially diluted antibody 1656 were mixed with each other on a 96-well round bottom plate. Each mixture was stood at 37° C. for 1 hour or more, or at room temperature overnight or longer. Then, agglutination of bacterial cells was judged.

As a result, the agglutination titer of the antibody 1656 was 64, in other words, agglutination was observed up to 64-fold dilution, and the agglutination titer per amount (μg) of IgG was 190. Meanwhile, an immunoglobulin preparation, Venilon, (50 mg/ml, TEIJIN PHARMA LIMITED), which was a control, did not cause the agglutination of the inactivated strain at all.

### (6) Opsonic Activity

### —Test 1—

The serotype E P. aeruginosa strain ATCC 29260 was cultured in a LB medium overnight. The bacterial culture was fixed with 4% paraformaldehyde, and suspended in a 1 mM solution of fluorescein-4-isothiocyanate (FITC) at room temperature for 1 hour to perform labeling. By a density gradient centrifugation method using a Mono-Poly resolving medium (DS Pharma Biomedical Co. Ltd.), human polymorphonuclear leukocytes (hereinafter, referred to as PMN) were purified from 50 ml of blood collected using citric acid from healthy donors, and were prepared to have a concentration of 5×10⁶ cells/ml. 20 μl of the serotype E specific antibody 1656 and the FITC-labeled P. aeruginosa strain (30 μl, 5×10⁶) were added in a 96-well round-bottom plate, and incubated at 37° C. for 15 minutes. Thereafter, as complements, baby rabbit serum (10 μl) and the PMN (40 μl, 2×10⁵ cells) were added, and the mixture was further incubated for 30 minutes to carry out phagocytosis. The plate was transferred onto ice, and thereby the reaction was stopped. The fluorescence of bacteria attaching to the cell surfaces was quenched by PBS containing 0.2% trypan blue (100 μl), and then the cells were fixed with 0.5% paraformaldehyde. Using a flow cytometer (BECKMAN COULTER), the fluorescence (mean fluorescence intensity, hereinafter abbreviated as MFI) of the cells was measured. The opsonic activity was calculated as a value obtained by subtracting the fluorescence intensity due to the intrinsic fluorescence of the PMN from the fluorescence intensity of PMN which incorporated the FITC-labeled P. aeruginosa strain.

As a result, for the serotype E strain ATCC 29260, the MFI value of a group to which no antibody was added was 0.32, and the MFI value of a group to which the anti-serotype E LPS antibody 1656 was added increased concentration-dependently, where the MFI value was 122.87 at 30 μg/ml, and the EC50 was 0.11 μg/ml. The MFI value of an immunoglobulin preparation, Venilon (TEIJIN PHARMA LIMITED), which was used as a control, was 97.77 at 1000 μg/ml.

The above-described results showed that the anti-serotype E LPS antibody 1656 had a strong opsonic activity against a strain of serotype E, which is clinically frequently encountered.

### —Test 2—

The serotype E P. aeruginosa strain ATCC 29260 was cultured on a Mueller-Hinton agar medium overnight. Then, 3 colonies were picked up therefrom, inoculated in a Luria-Bertani culture medium, and cultured at 37° C. for 16 hours with shaking (180 rpm). The culture medium was subjected to centrifugation (2,000×g, 10 minutes, at room temperature). The resultant material was washed once with phosphate-buffered saline (PBS), and then suspended in a 1 mM solution of fluorescein-4-isothiocyanate (FITC) at room temperature for 1 hour to perform labeling. By a density gradient centrifugation method using a Mono-Poly resolving medium (DS Pharma Biomedical Co. Ltd.), human polymorphonuclear leukocytes (hereinafter, referred to as PMN) were purified from 50 ml of blood collected using citric acid from healthy donors, and were prepared to have a concentration of 5×10⁶ cells/ml. 20 μl of the anti-serotype E LPS antibody 1640 and the FITC-labeled P. aeruginosa strain (30 μl, 5×10⁶) were added in a 96-well round-bottom plate, and incubated at 37° C. for 15 minutes. Thereafter, as complements, baby rabbit serum (10 μl) and the PMN (40 μl, 2×10⁵ cells) were added, and the mixture was further incubated for 30 minutes to carry out phagocytosis. The plate was transferred onto ice, and thereby the reaction was stopped. The fluorescence of bacteria attaching to the cell surfaces was quenched by PBS containing 0.2% trypan blue (100 μl), and then the cells were fixed with 0.5% paraformaldehyde. Using a flow cytometer (BECKMAN COULTER), the fluorescence (mean fluorescence intensity, hereinafter abbreviated as MFI) of the cells was measured. The opsonic activity was calculated as a value obtained by subtracting the fluorescence intensity due to the intrinsic fluorescence of the PMN from the fluorescence intensity of PMN which incorporated the FITC-labeled P. aeruginosa strain.

As a result, for the serotype E P. aeruginosa strain ATCC 29260, the MFI value of a group to which no antibody was added was 0.44, and the MFI value of a group to which the antibody 1640 was added increased concentration-dependently, where the MFI value was 58.37 at 30 μg/ml, and the EC50 was 0.64 μg/ml. The MFI value of an immunoglobulin preparation, Venilon (TEIJIN PHARMA LIMITED), which was used as a control, was 27.07 at 1000 μg/ml.

The above-described results showed that the anti-serotype E LPS antibody 1640 had an opsonic activity against a P. aeruginosa strain of serotype E.

### (7) Effect on Systemic Infection Model 1

Neutropenic mice were prepared as follows. Cyclophosphamide (Sigma-Aldrich) was intraperitoneally injected into each 6-week-old BALB/c male mouse (Charles river laboratories Japan, inc., n=6) at 125 mg/kg three times in total on days −5, −2 and 0, where the day of infection was designated as day 0. Thereby, neutrophils in the peripheral blood were decreased. Into the mouse, the ATCC 29260 strain (serotype E/O11) suspended in 250 μl of saline was inoculated intraperitoneally at 1.8×10 cfu/mouse (approximately 46 LD50), to thereby induce a systemic infection. Immediately thereafter, the anti-serotype E LPS antibody 1640 was administered via tail vein at 200 μl/mouse, and a protective activity against the infection was judged on the basis of the survival thereof 7 days after the inoculation. As a result, the survival rates, on day after the infection, of control groups to which an immunoglobulin preparation, Venilon (TEIJIN PHARMA LIMITED), was administered at 5, 50, 500 and 2500 μg/mouse were 0, 16.7, 33.3 and 66.7%, respectively, and the ED50 was estimated to be 985.22 μg/mouse. In contrast, the survival rates, on day 7 after the infection, of groups to which the anti-serotype E LPS antibody 1640 was administered at 5, 10, 20, 50, 100 and 250 μg/mouse were 0, 50, 100, 16.7, 66.7 and 100%, respectively, showing a strong protective activity against the infection, and the ED50 was estimated to be 23.06 μg/mouse.

### (8) Effect on Systemic Infection Model 2

Neutropenic mice were prepared as follows. Cyclophosphamide (Sigma-Aldrich) was intraperitoneally injected into each 6-week-old BALB/c male mouse (Charles river laboratories Japan, inc., n=6) at 125 mg/kg three times in total on days −5, −2 and 0, where the day of infection was designated as day 0. Thereby, neutrophils in the peripheral blood were decreased. The ATCC 29260 strain (serotype E/O11) was inoculated intraperitoneally at 1.475×10³ cfu/mouse (approximately 38 LD50), to thereby induce a systemic infection. Immediately thereafter, a sample was administered via tail vein at 200 μl/mouse, and a protective activity against the infection was judged on the basis of the survival thereof 7 days after the inoculation. Asa result, the survival rates, on day 7 after the infection, of control groups to which an immunoglobulin preparation, Venilon (TEIJIN PHARMA LIMITED), was administered at 40, 200, 1000 and 5000 μg/mouse were 0, 16.7, 16.7 and 83.3%, respectively, and the ED50 was estimated to be 1779.93 μg/mouse. The survival rates, on day 7 after the infection, of groups to which an anti-PcrV antibody M166 was administered at 1.6, 8, 40, 200 and 400 μg/mouse were 0, 0, 0, 50 and 16.7%, respectively, and the ED50 was estimated to be 714.91 μg/mouse or more. In contrast, the survival rates, on day 7 after the infection, of groups to which the anti-serotype E LPS antibody 1656 was administered at 0.32, 1.6, 8, 40 and 200 μg/mouse were 0, 50, 50, 66.7 and 66.7%, showing a strong protective activity against the infection, and the ED50 was estimated to be 12.21 μg/mouse.

### (9) Effect on Systemic Infection Model 3

Neutropenic mice were prepared as follows.

Cyclophosphamide (hereinafter referred to as CY, Sigma-Aldrich) was intraperitoneally injected into each 6-week-old BALB/c male mouse (Charles River Laboratories Japan, Inc., n=6) at 125 mg/kg three times in total on days −5, −2 and 0, where the day of infection was designated as day 0. Thereby, neutrophils in the peripheral blood were decreased. Into the mouse, the MSC 06120 strain (serotype E/O11, MDRP) suspended in 250 of saline was inoculated intraperitoneally at 1.575×10⁴ cfu/mouse (>1260 LD50), to thereby induce a systemic infection. Immediately after the inoculation, a sample was administered via tail vein at 200 μl/mouse, and a protective activity against the infection was evaluated on the basis of the survival thereof 7 days after the inoculation. As a result, the survival rates, on day 7 after the infection, of control groups to which an immunoglobulin preparation, Venilon (TEIJIN PHARMA LIMITED), was administered at 40, 200, 1000, and 5000 μg/mouse were 16.7, 0, 33.3, and 83.3%, respectively, and the ED50 was estimated to be 1498.38 μg/mouse. The survival rates, on day 7 after the infection, of groups to which an anti-PcrV antibody M166 was administered at 1.6, 8, 40, and 200 μg/mouse were 0, 0, 0, and 16.7%, respectively, and the ED50 was estimated to be 257.71 μg/mouse. In contrast, the survival rates, on day 7 after the infection, of groups to which the antibody 1656 was administered at 0.32, 1.6, 8, and 40 μg/mouse were 16.7, 50, 16.7, and 83.3%, respectively, showing a strong protective activity against the infection, and the ED50 was estimated to be 8.05 μg/mouse.

### (10) Effect on Pulmonary Infection Model

Evaluation on a normal mouse acute pulmonary infection model was made as follows. 5-week-old BALB/c male mice (Charles River laboratories Japan, inc., n=6) were used. The ATCC 29260 strain (serotype E/O11) suspended in saline was nasally inoculated to the mice at 2.64×10⁵ CFU/20 μl/mouse (approximately 13 LD50) under ketamine/xylazine anesthesia. Immediately thereafter, a sample was administered via tail vein at 200 μl/mouse, and a protective activity against the infection was judged on the basis of the survival thereof 7 days after the inoculation. As a result, all mice in an infected control group were dead within 2 days after the infection. The survival rates, on day 7 after the infection, of positive control groups to which an immunoglobulin preparation, Venilon (TEIJIN PHARMA LIMITED), was administered at 100, 500 or 2500 μg/mouse were 33.3, 83.3 and 100%, respectively, and the ED50 was estimated to be 163.53 μg/mouse. The survival rates, on day 7 after the infection, of groups to which an anti-PcrV antibody M166 was administered at 0.16, 0.8, 4 and 20 μg/mouse were 0, 0, 16.7 and 83.3%, respectively, and the ED50 was estimated to be 8.99 μg/mouse. In contrast, the survival rates, on day 7 after the infection, of groups to which the anti-serotype E LPS antibody 1640 was administered at 0.032, 0.08, 0.16, 0.8, 4 and 20 μg/mouse were 0, 33.3, 16.7, 100, 100 and 100%, respectively, showing a strong protective activity against the infection, and the ED50 was estimated to be 0.19 μg/mouse. Meanwhile, the survival rates, on day 7 after the infection, of groups to which the anti-serotype E LPS antibody 1656 was administered at 0.032, 0.08, 0.16, 0.8, 4 and 20 μg/mouse were 0, 0, 50, 100, 100 and 100%, respectively, showing a strong protective activity against the infection, and the ED50 was 0.16 μg/mouse.

### (11) Effect on Pulmonary Infection Model 2

A protection effect against infection of post-infection administration of an antibody was evaluated using a normal mouse acute pulmonary infection model. Specifically, 5-week-old BALB/c male mice (Charles River Laboratories Japan, Inc., n=12) were used. The ATCC 29260 strain (serotype E/O11) suspended in saline was nasally inoculated to each mouse at 2.84 or 4.49×10⁵ CFU/20 μl/mouse (approximately 14 or 22 LD50) under ketamine/xylazine anesthesia. Eight hours later, a sample was administered via tail vein at 200 μl/mouse, and a protective activity against the infection was evaluated on the basis of the survival thereof 7 days after the inoculation. Asa result, the survival rates, on day 7 after the infection, of control groups to which an immunoglobulin preparation, Venilon (TEIJIN PHARMA LIMITED), was administered at 100, 500, and 2500 μg/mouse were 0, 25, and 33.3%, respectively, and the ED50 was estimated to be 4650.69 μg/mouse. In contrast, the survival rates, on day 7 after the infection, of groups to which the antibody 1656 was administered at 0.16, 0.8, 4, and 20 μg/mouse were 8.3, 58.3, 83.3, and 100%, respectively, and the ED50 was estimated to be 0.80 μg/mouse. The post-infection administration also exhibited a strong protective activity against the infection.

The lungs were observed histopathologically. As a result, 24 hours after the infection, histopathological findings of hemorrhagic and suppurative pneumonia such as neutrophil infiltration to the pulmonary alveoli, vascular walls, bronchi, and bronchioles, and intense edema around blood vessels were observed in the infection control group and the Venilon-treated group. In contrast, in the 1656 antibody-treated group, neutrophil infiltration to the bronchi and blood vessels was reduced, and the pneumonia was alleviated. In addition, the presence of macrophages was observed, indicating that transition to a healing stage occurred at an early stage. Meanwhile, on the day 8 after the infection, the pneumonia was cured in the 1656 antibody-treated group to such an extent that the pneumonia was not observed any more.

### (12) Effect on Pulmonary Infection Model 3

A protection effect against infection was evaluated using a normal mouse acute pulmonary infection model induced by MDRP. Specifically, 5-week-old BALB/c male mice (Charles River Laboratories Japan, Inc., n=6) were used. The MSC 06120 strain (serotype E/O11, MDRP) suspended in saline was nasally inoculated to each mouse at 4.26×10⁶ CFU/20 μl/mouse (approximately 4.2 LD50) under ketamine/xylazine anesthesia. Immediately thereafter, a sample was administered via tail vein at 200 μl/mouse, and a protective activity against the infection was evaluated on the basis of the survival thereof 7 days after the inoculation. As a result, the survival rates, on day 7 after the infection, of control groups to which an immunoglobulin preparation, Venilon (TEIJIN PHARMA LIMITED), was administered at 40, 200, 1000, and 5000 μg/mouse were 0, 0, 0, and 33.3%, respectively, and the ED50 was estimated to be 5000 μg/mouse. The survival rates, on day 7 after the infection, of groups to which an anti-PcrV antibody M166 was administered at 1.6, 8, and 40 μg/mouse were 0, 0, and 16.7%, respectively, and the ED50 was estimated to be >40 μg/mouse. In contrast, the survival rates, on day 7 after the infection, of groups to which the antibody 1656 was administered at 0.32, 1.6, 8, 40, and 200 μg/mouse were 0, 16.7, 66.7, 83.3, and 100%, respectively, showing a strong protective activity against the infection, and the ED50 was estimated to be 6.31 μg/mouse.

### (13) Effect on Pulmonary Infection Model 4

A protection effect against infection of post-infection administration of an antibody was evaluated using a normal mouse acute pulmonary infection model induced by MDRP. Specifically, 5-week-old BALB/c male mice (Charles River Laboratories Japan, Inc., n=6 or 12) were used. The MSC 06120 strain (serotypeE/O11, MDRP) suspended in saline was nasally inoculated to each mouse at 2.90 or 3.78×10⁶ CFU/20 μl/mouse (approximately 2.9 or 3.7 LD50) under ketamine/xylazine anesthesia. Eight hours later, a sample was administered via tail vein at 200 μl/mouse, and a protective activity against the infection was evaluated on the basis of the survival thereof 7 days after the inoculation. As a result, the survival rates, on day 7 after the infection, of control groups to which an immunoglobulin preparation, Venilon (TEIJIN PHARMA LIMITED), was administered at 40, 200, 1000, and 5000 μg/mouse were 0, 8.3, 25, and 0%, respectively, and the ED50 was estimated to be >5000 μg/mouse. The survival rates, on day 7 after the infection, of groups to which an anti-PcrV antibody M166 was administered at 1.6, 8, 40, and 200 μg/mouse were 0, 0, 8.3, and 0%, respectively, and the ED50 was estimated to be >200 μg/mouse. In contrast, the survival rates, on day 7 after the infection, of groups to which the antibody 1656 was administered at 1.6, 8, 40, and 200 μg/mouse were 25, 8.3, 58.3, and 58.3%, respectively, and the ED50 was estimated to be 70.22 μg/mouse. The post-infection administration also exhibited a strong protective activity against the infection.

### (14) Effect on Burn Wound Infection Model 1

A protection effect against infection was evaluated using a normal mouse burn wound infection model. Specifically, 7-week-old C57BL/6J male mice (Charles River Laboratories Japan, Inc., n=8) were used. On the day before the infection, the back of each mouse was shaved under isoflurane anesthesia by use of an animal electric shaver (National) and a hair removal cream (Kanebo Cosmetics Inc.). On the day of infection, the shaved back (2×3 cm) was brought into contact with hot water at 87° C. for eight seconds under ketamine/xylazine anesthesia, and immediately thereafter soaked in sterile water at room temperature for eight seconds. Then, 0.5 ml of saline was administered to the abdominal cavity, and then the ATCC 29260 strain (serotype E/O11) suspended in saline was inoculated to the subcutaneous tissue at the wound site at 0.86 or 1.0×10⁴ CFU/100 μl/mouse (approximately 81 or 94 LD50), to thereby induce infection. Immediately thereafter, a sample was administered via tail vein at 200 μl/mouse, and a protective activity against the infection was evaluated on the basis of the survival thereof 14 days after the inoculation. Asa result, the survival rates, on day 14 after the infection, of control groups to which an immunoglobulin preparation, Venilon (TEIJIN PHARMA LIMITED), was administered at 40, 200, 1000, and 5000 μg/mouse were 37.5, 87.5, 87.5, and 87.5%, respectively, and the ED50 was estimated to be 37.825 μg/mouse. The survival rates, on day 14 after the infection, of groups to which an anti-PcrV antibody M166 was administered at 0.8, 4, 20, and 100 μg/mouse were 12.5, 50, 37.5, and 50%, respectively, and the ED50 was estimated to be 63.30 μg/mouse. In contrast, the survival rates, on day 14 after the infection, of groups to which the antibody 1656 was administered at 0.0064, 0.032, 0.16, and 0.8 μg/mouse were 37.5, 50, 100, and 87.5%, respectively, showing a strong protective activity against the infection, and the ED50 was estimated to be 0.015 μg/mouse.

### (15) Effects on Burn Wound Infection Model 2

A protection effect against infection of post-infection administration of an antibody was evaluated using a normal mouse burn wound infection model. Specifically, 7-week-old C57BL/6J male mice (Charles River Laboratories Japan, Inc., n=8 to 10) were used. On the day before the infection, the back of each mouse was shaved under isoflurane anesthesia by use of an animal electric shaver (National) and a hair removal cream (Kanebo Cosmetics Inc.). On the day of infection, the shaved back (2×3 cm) was brought into contact with hot water at 87° C. for eight seconds under ketamine/xylazine anesthesia, and immediately thereafter soaked in sterile water at room temperature for eight seconds. Then, 0.5 ml of saline was administered to the abdominal cavity, and then the ATCC 29260 strain (serotype E/O11) suspended in saline was inoculated to the subcutaneous tissue at the wound site at 1.23 or 1.62×10⁴ CFU/100 μl/mouse (approximately 116 or 153 LD50), to thereby induce infection. Twenty-five hours later, a sample was administered via tail vein at 200 μl/mouse, and a protective activity against the infection was evaluated on the basis of the survival thereof 14 days after the inoculation. As a result, the survival rates, on day 14 after the infection, of control groups to which an immunoglobulin preparation, Venilon (TEIJIN PHARMA LIMITED), was administered at 200, 1000, and 5000 μg/mouse were 0, 62.5, and 87.5%, respectively, and the ED50 was estimated to be 1059.51 μg/mouse. The survival rates, on day 14 after the infection, of groups to which an anti-PcrV antibody M166 was administered at 4, 20, and 100 μg/mouse were 12.5, 12.5, and 22.2%, respectively, and the ED50 was estimated to be >100 μg/mouse. In contrast, the survival rates, on day 14 after the infection, of groups to which the antibody 1656 was administered at 0.16, 0.8, 4, and 20 μg/mouse were 33.3, 66.7, 88.9, and 88.9%, respectively, and the ED50 was estimated to be 0.35 μg/mouse. The post-infection administration of the antibody also exhibited a strong protective activity against the infection.

### Example 3

### Combination of Anti-Serotype E LPS Antibody 1656 and Broadly Reactive Anti-LPS Antibody 2459

### —Effect on Pulmonary Infection Model—

An effect of combined use of the anti-serotype E LPS antibody 1656 and the broadly reactive anti-LPS antibody 2459 (the antibody which recognizes A-band LPS of lipopolysaccharides of P. aeruginosa, and which substantially binds to surfaces of at least P. aeruginosa strains of serotype A, B, C, D, E, G, H, I, M, N, 018 and 019; amino acid sequences of light chain CDRs 1 to 3 described in SEQ ID NOs: 17 to 19, amino acid sequences of heavy chain CDRs 1 to 3 described in SEQ ID NOs: 20 to 22, an amino acid sequence of light chain variable region described in SEQ ID NO:23, an amino acid sequences of Heavy chain variable region described in SEQ ID NO:24, a base sequence of light chain variable region described in SEQ ID NO:29, a base sequence of Heavy chain variable region described in SEQ ID NO:30.) was evaluated using a normal mouse acute pulmonary infection model. Specifically, 5-week-old BALB/c male mice (Charles River laboratories Japan, inc. n=6) were used. The ATCC 29260 strain (serotype E/O11) suspended in saline was nasally inoculated to the mice at 3.34×10⁵ CFU/20 μl/mouse (approximately 9 LD₅₀) under ketamine/xylazine anesthesia. Immediately thereafter, a sample was administered via tail vein at 200 μl/mouse, and a protective activity against the infection was judged on the basis of the survival thereof 7 days after the inoculation. As a result, all mice in an infected control group were dead within 3 days after the infection. The survival rates, on day 7 after the infection, of groups to which the antibody 2459 was administered at 0.2, 0.4 and 0.8 μg/mouse were 0, 16.7 and 0%, respectively. Hence, the antibody 2459 was ineffective. The survival rate, on day 7 after the infection, of a group to which the anti-serotype E LPS antibody 1656 was administered at 0.2 μg/mouse was 33.3%. In contrast, surprisingly, the survival rates, on day 7 after the infection, of groups to which the both were co-administered, that is, groups to which combinations of the antibody 2459 at 0.2, 0.4 and 0.8 μg/mouse, respectively, with the antibody 1656 at 0.2 μg/mouse were administered, respectively, were 66.7, 83.3 and 100%, respectively, showing improvement which was dependent on the dose of the antibody 2459. It was found out that a combined use of the anti-serotype E LPS antibody 1656 and the broadly reactive anti-LPS antibody 2459 provided a synergistic effect.

### —Effect in SPR Measurement—

In order to confirm the effect of the combined use of the anti-serotype E LPS antibody 1656 and the broadly reactive anti-LPS antibody 2459, surface plasmon resonance (SPR) measurement was performed by use of a liposome containing 1,2-dimyristoyl-sn-glycero-3-phosphocholine (DMPC) (Sigma, P7331) as a matrix phospholipid and containing the LPS E/O11 which was obtained from the P. aeruginosa strain ATCC 29260 and which was prepared in Example 2. The SPR measurement is known as a method which allows real-time analysis of molecular interactions without labeling, and has been widely used for analysis of antigen-antibody reactions.

The measurement was performed by using a ProteOn XPR 36 system (Bio-Rad) as an SPR measurement apparatus, a ProteOn GLM chip (Bio-Rad, 176-5012) as a sensor chip, and a PBS buffer pH 7.4 (Sigma, D5652) as a mobile phase.

DMPC was dissolved, so as to be 10 mM, in the PBS buffer or a PBS buffer containing the LPS E/O₁₁ at 0.4 mg/ml. After freeze-thaw operation was performed five times, each mixture was passed through a 100-nm filter 21 times using a Mini-Extruder (Anti Polar Lipids, Inc), and thereby a homogeneous liposome was prepared.

To create hydrophobicity necessary for immobilization of the liposome, undecylamine (Sigma, 94200) was dissolved in dimethyl sulfoxide (nacalai tesque, 13445-74) at 1%, and the solution was diluted 20-fold with a ProteOn Acetate buffer pH 5.0 (Bio-Rad, 176-2122). Then, the undecylamine was immobilized onto the sensor chip by use of a ProteOn amine coupling kit (Bio-Rad, 176-2410). Onto the chip onto which undecylamine was immobilized, the liposome containing the LPS E/O11 as a ligand and the liposome containing no LPS as a negative control were immobilized. As analytes, the antibody 2459 and the antibody 1656 prepared in Example 2 were used, which were prepared to have the same concentration of 200 nM using the mobile phase, for use in the measurement. The antibody 2459 or the antibody 1656 was injected to the sensor chip, with the flow rate being set to 30 μl/minutes, and the binding time being set to 2 minutes. Thereafter, the same antibody as the injected antibody, or the other antibody was additionally injected in the same manner. Double reference was performed on the obtained sensor grams by subtracting the value obtained with adsorption to the liposome containing no LPS and the value obtained with the mobile phase alone (the concentration of the antibody if j). Thus, evaluation was made by using only specific binding to the LPS E/O11.

FIG. 3 shows the obtained sensor grams. Even after the anti-serotype E LPS antibody 1656, or the broadly reactive anti-LPS antibody 2459 bound, it was observed that the other one of the antibodies bound in the same manner as in the case of the other antibody alone.

These results showed that the antibody 1656 recognized an epitope different from that recognized by the antibody2459, and the antibody 1656 and the antibody 2459 was capable of simultaneous binding.

### INDUSTRIAL APPLICABILITY

An antibody of the present invention has an excellent antibacterial activity against P. aeruginosa, and hence can be used for treatment or prevention of P. aeruginosa infections. Antibodies of the present invention can be combined to form a polyclonal preparation which exhibits a potent antibacterial activity against a broad range of clinically isolated strains. Moreover, the antibody of the present invention is a human antibody, and hence is highly safe. Accordingly, the antibody of the present invention is extremely useful for medical care. Furthermore, the monoclonal antibody of the present invention can be applied for diagnosis of P. aeruginosa infections, detection or screening of P. aeruginosa strains of various serotypes, and the like.

1. An antibody which recognizes B-band LPS of lipopolysaccharides of P. aeruginosa, and which substantially binds to a surface of a P. aeruginosa strain of serotype E, but does not substantially binds to any one of surfaces of P. aeruginosa strains of serotype A, B, C, D, F, G, H, I and M.

2. The antibody according to claim 1, which has an opsonic activity against a P. aeruginosa strain of serotype E.

3. The antibody according to claim 2, wherein an EC50 of an opsonic activity against a P. aeruginosa strain identified by ATCC 29260 is 1 μg/ml or less.

4. The antibody according to claim 1, which has an agglutination activity against a P. aeruginosa strain of serotype E.

5. The antibody according to claim 4, wherein an agglutination titer per amount (μg) of IgG against a P. aeruginosa strain identified by ATCC 29260 is 100 or more.

6. The antibody according to claim 1, which has an antibacterial effect against a systemic infection with a P. aeruginosa strain of serotype E.

7. The antibody according to claim 6, wherein an ED50 of an antibacterial effect on a neutropenic mouse model of systemic infection with a P. aeruginosa strain identified by ATCC 29260 is not more than 1/30 of that of Venilon.

8. The antibody according to claim 1, which has an antibacterial effect against a pulmonary infection with a P. aeruginosa strain of serotype E.

9. The antibody according to claim 8, wherein an antibacterial effect on a mouse model of pulmonary infection with a P. aeruginosa strain identified by ATCC 29260 has at least one property selected from the following group:
(a) upon administration of the antibody to a mouse immediately after the inoculation with a P. aeruginosa strain identified by ATCC 29260 to the mouse, an ED50 of the antibacterial effect on the mouse is not more than 1/500 of that of Venilon; and
(b) upon administration of the antibody to a mouse 8 hours after the inoculation with a P. aeruginosa strain identified by ATCC 29260 to the mouse, an ED50 of the antibacterial effect on the mouse is not more than 1/3000 of that of Venilon.

10. The antibody according to claim 1, which has an antibacterial effect against a burn wound infection with a P. aeruginosa strain of serotype E.

11. The antibody according to claim 10, wherein an antibacterial effect on a mouse model of burn wound infection with a P. aeruginosa strain identified by ATCC 29260 has at least one property selected from the following group:
(a) upon administration of the antibody to a mouse immediately after the inoculation with a P. aeruginosa strain identified by ATCC 29260 to the mouse, an ED50 of the antibacterial effect on the mouse is not more than 1/1500 of that of Venilon; and
(b) upon administration of the antibody to a mouse 25 hours after the inoculation with a P. aeruginosa strain identified by ATCC 29260 to the mouse, an ED50 of the antibacterial effect on the mouse is not more than 1/2000 of that of Venilon.

12. The antibody which has any one of the following features (a) and (b):
(a) comprising
a light chain variable region including amino acid sequences described in SEQ ID NOs: 1 to 3 or the amino acid sequences described in SEQ ID NOs: 1 to 3 in at least one of which one or more amino acids are substituted, deleted, added, and/or inserted, and
a heavy chain variable region including amino acid sequences described in SEQ ID NOs: 4 to 6 or the amino acid sequences described in SEQ ID NOs: 4 to 6 in at least one of which one or more amino acids are substituted, deleted, added, and/or inserted; and
(b) comprising
a light chain variable region including amino acid sequences described in SEQ ID NOs: 9 to 11 or the amino acid sequences described in SEQ ID NOs: 9 to 11 in at least one of which one or more amino acids are substituted, deleted, added, and/or inserted, and
a heavy chain variable region including amino acid sequences described in SEQ ID NOs: 12 to 14 or the amino acid sequences described in SEQ ID NOs: 12 to 14 in at least one of which one or more amino acids are substituted, deleted, added, and/or inserted.

13. The antibody which has any one of the following features (a) and (b):
(a) comprising
a light chain variable region including an amino acid sequence described in SEQ ID NO: 7 or the amino acid sequence described in SEQ ID NO: 7 in which one or more amino acids are substituted, deleted, added, and/or inserted, and
a heavy chain variable region including an amino acid sequence described in SEQ ID NO: 8 or the amino acid sequence described in SEQ ID NO: 8 in which one or more amino acids are substituted, deleted, added, and/or inserted; and
(b) comprising
a light chain variable region including an amino acid sequence described in SEQ ID NO: 15 or the amino acid sequence described in SEQ ID NO: 15 in which one or more amino acids are substituted, deleted, added, and/or inserted, and
a heavy chain variable region including an amino acid sequence described in SEQ ID NO: 16 or the amino acid sequence described in SEQ ID NO: 16 in which one or more amino acids are substituted, deleted, added, and/or inserted.

14. A peptide comprising a light chain or a light chain variable region of the antibody, the peptide having any one of the following features (a) and (b):
(a) comprising amino acid sequences described in SEQ ID NOs: 1 to 3 or the amino acid sequences described in SEQ ID NOs: 1 to 3 in at least one of which one or more amino acids are substituted, deleted, added, and/or inserted; and
(b) comprising amino acid sequences described in SEQ ID NOs: 9 to 11 or the amino acid sequences described in SEQ ID NOs: 9 to 11 in at least one of which one or more amino acids are substituted, deleted, added, and/or inserted.

15. A peptide comprising a light chain or a light chain variable region of the antibody, the peptide having any one of the following features (a) and (b):
(a) comprising an amino acid sequence described in SEQ ID NO: 7 or the amino acid sequence described in SEQ ID NO: 7 in which one or more amino acids are substituted, deleted, added, and/or inserted; and
(b) comprising an amino acid sequence described in SEQ ID NO: 15 or the amino acid sequence described in SEQ ID NO: 15 in which one or more amino acids are substituted, deleted, added, and/or inserted.

16. A peptide comprising a heavy chain or a heavy chain variable region of the antibody, which has any one of the following features (a) and (b):
(a) comprising amino acid sequences described in SEQ ID NOs: 4 to 6 or the amino acid sequences described in SEQ ID NOs: 4 to 6 in at least one of which one or more amino acids are substituted, deleted, added, and/or inserted; and
(b) comprising amino acid sequences described in SEQ ID NOs: 12 to 14 or the amino acid sequences described in SEQ ID NOs: 12 to 14 in at least one of which one or more amino acids are substituted, deleted, added, and/or inserted.

17. A peptide comprising a heavy chain or a heavy chain variable region of the antibody, which has any one of the following features (a) and (b):
(a) comprising an amino acid sequence described in SEQ ID NO: 8 or the amino acid sequence described in SEQ ID NO: 8 in which one or more amino acids are substituted, deleted, added, and/or inserted; and
(b) comprising an amino acid sequence described in SEQ ID NO: 16 or the amino acid sequence described in SEQ ID NO: 16 in which one or more amino acids are substituted, deleted, added, and/or inserted.

18. An antibody which binds to an epitope, in B-band LPS of lipopolysaccharides of a P. aeruginosa strain of serotype E, of an antibody described in any one of the following (a) and (b):
(a) an antibody comprising a light chain variable region including an amino acid sequence described in SEQ ID NO: 7, and a heavy chain variable region including an amino acid sequence described in SEQ ID NO: 8; and
(b) an antibody comprising a light chain variable region including an amino acid sequence described in SEQ ID NO: 15 and a heavy chain variable region including an amino acid sequence described in SEQ ID NO: 16.

19. A DNA which codes the antibody or the peptide according to claim 1.

20. A hybridoma which produces the antibody according to claim 1.

21. A pharmaceutical composition for a disease associated with P. aeruginosa, the pharmaceutical composition comprising:
the antibody according to claim 1; and optionally
at least one pharmaceutically acceptable carrier and/or diluent.

22. The pharmaceutical composition according to claim 21, wherein the disease associated with P. aeruginosa is a systemic infectious disease caused by a P. aeruginosa infection.

23. The pharmaceutical composition according to claim 21, wherein the disease associated with P. aeruginosa is a pulmonary infectious disease caused by a P. aeruginosa infection.

24. The pharmaceutical composition according to claim 21, wherein the disease associated with P. aeruginosa is a burn wound infectious disease caused by a P. aeruginosa infection.

25. A diagnostic agent for detection of P. aeruginosa, the diagnostic agent comprising: the antibody according to claim 1.

26. A kit for detection of P. aeruginosa, the kit comprising: the antibody according to claim 1.
